(19) United States
(12) Patent Application Publication
(10) Pub. No.: US 2023/0181798 A1
(43) Pub. Date: Jun. 15, 2023

(54) BIO-NANOCOMPOSITE FOR BONE TISSUE ENGINEERING

(71) Applicant: The Governors of the University of Alberta, Edmonton (CA)

(72) Inventors: Mohammad Reza VAKILI, Edmonton (CA); Afsaneh LAVASANIFAR, Edmonton (CA); Behzad AHVAZI, Edmonton (CA)

(21) Appl. No.: 18/064,578

(22) Filed: Dec. 12, 2022

Related U.S. Application Data
(60) Provisional application No. 63/288,913, filed on Dec. 13, 2021.

(51) Int. Cl. A61L 27/46; A61L 27/56; A61L 27/38

(52) U.S. Cl. CPC A61L 27/46; A61L 27/56; A61L 27/3821; A61L 27/3817; A61L 2400/12; A61L 2430/02; A61L 2430/06; A61L 2300/414

(57) ABSTRACT

This disclosure describes bone tissues engineered from a casted bio-nanocomposite comprising chitosan crosslinked with citric acid to cellulose nanocrystals (CNC) where the amount of CNC used was as high as 29.4%. The nanocomposite showed proper characteristics of a bone mimicking structure. Different layers of the bio-nanocomposite showed an average pore size of greater than 26 micrometers in diameter; a porosity of about 90%, firm structure, maximum bioactivity as measured by deposition of calcium phosphate from simulated body fluid (SBF) solution (gaining weight more than 20% after 3 days), decreased rate of in vitro degradation in PBS (7-60 days), about 10% after 7 days, and acceptable bone cell viability (greater than 80%) in 2D and 3D cultures. The compression modulus of the bio-nanocomposites increased about 4 times and exhibited very small changes in size during the swelling process compared to control.

### RELATED APPLICATIONS

This application claims priority under 35 U.S.C. § 119(e) to U.S. Provisional Patent Application No. 63/288,913, filed Dec. 13, 2021, which is incorporated herein by reference.

### BACKGROUND OF THE INVENTION

Half of the chronic diseases in people over 50 years old is related to bone associate diseases. Certain bone diseases and injuries lead to bone defects either small or large defects. Most of the large segmental bone defects are caused by a traffic accident, non-union fracture, infection, trauma, tumor, and congenital etiology. Bone tissue can heal its defects with a limited regeneration capacity. While small segmental bone damages can be healed by the self-healing process of the bone tissue, large segmental bone defect repair could not be accomplished by the bone tissue itself (typically >2 cm depending on the defected site). Therefore, bone healing defect remains a clinically challenging point in orthopaedics.

Bone tissue engineering attributes to repair and heal the large segmental damages. One of the promising goals of bone tissue engineering is to design and fabricate regenerative biomaterial scaffolds to fill the site of large segmental bone defects. Regenerative biomaterial scaffolds perform as temporary artificial environments specified to facilitate bone cell proliferation as well as cell differentiation to develop and grow natural bone tissues.

The problem is biodegradable nanocomposites for bone repair are lacking mechanical properties needed for bone regeneration. Therefore, improvement in the formulation polymeric matrices is needed to provide biodegradable scaffolds that are biocompatible and have sufficient mechanical strength for cartilage tissue engineering.

### SUMMARY

A series of bio-nanocomposites were fabricated by chitosan (CS), cellulose nanocrystals (CNC), hydroxylapatite (HA). Chitosan was chosen as a matrix of the bio-nanocomposite scaffolds, HA was employed as one of the main components in the bio-nanocomposite formulations to mimic native bone tissue, and CNC is included to provide porosity and mechanical strength.

This disclosure describes 8 bio-nanocomposites that were fabricated, characterized, and evaluated for bone tissue engineering application. Among all structures, bio-nanocomposites prepared by the freeze-drying method using the highest amount of CNC with crosslinking by citric acid, namely SC5, show proper characteristics as a bone mimicking structure. SC5 scaffold includes different cylindrical layers of the bio-nanocomposite with an average pore size of >26 micrometers in width; a porosity of ˜90%, firm structure, maximum bioactivity as measured by deposition of calcium phosphate from SBF solution (gaining weight more than 20% after 3 days), 10% in vitro degradation in PBS in 7 days, and bone cells viability >80% in 2D and 3D cultures. Both Mg63 and MC3TC bone cells were accommodated, proliferated, and migrated in the pores of SC5 bio-nanocomposites in 3D culture. The mechanical property of the modified scaffold promoted by CNC and the crosslinker. The compressive modulus of SC5 (50 MPa) increased about 4 times compared to the unmodified scaffolds counterpart (S5). SC5 exhibited very small changes in size during the swelling process compared to S5.

Accordingly, this disclosure provides a bio-nanocomposite comprising chitosan (CS), cellulose nanocrystals (CNC) and apatite (XA) wherein the mass ratio CS:CNC is about 0.5:1 to about 10:1, CS is crosslinked to CNC via a linker to form a polymer matrix (PM), and XA is uniformly dispersed in the polymer matrix wherein the mass ratio of XA:PM is about 1:1 to about 1.5:1.

Also, this disclosure provides a method for forming a bio-nanocomposite artificial matrix for bone or cartilage comprising:

a) contacting chitosan (CS), cellulose nanocrystals (CNC), sintered hydroxylapatite (HA) and an aqueous solution at about pH 5 to about pH6 to form a mixture;

b) freezing the mixture in a cast and lyophilizing the frozen mixture to form a scaffold;

c) contacting the scaffold, citric acid, and an alcohol; and

d) removing excess citric acid and the alcohol;

wherein the mass ratio of CS:CNC is about 0.5:1 to about 10:1, CS is crosslinked to CNC via citric acid to form a polymer matrix (PM), and the mass ratio of HA:PM is about 1:1 to about 1.5:1; and wherein HA is uniformly dispersed in the polymer matrix and the artificial matrix is thereby formed.

Additionally, this disclosure provides a method for regenerating bone tissue comprising contacting the bio-nanocomposite described above and bone cells or cartilage under suitable physiological conditions wherein bone tissue is thereby regenerated.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A. SEM images of S0 Scaffold.

FIG. 1B. SEM images of S1 Scaffold.

FIG. 1C. SEM images of S3 Scaffold.

FIG. 1D. SEM images of S5 Scaffold.

FIG. 2. Pore size measured by SEM vs CNC amount in the bio-nanocomposite; P=0.00045895, one-way ANOVA.

FIG. 3. SEM images for SC5, with two magnifications (a-52X and b-200X).

FIG. 4. Kinetic swelling of SC1, SC3 and SC5 (n=3).

FIG. 5. Equilibrium swelling of SC1, SC3 and SC5 (n=3).

FIG. 6. SC5 scaffold before (top) swelling and (bottom) S5 scaffold before swelling.

FIG. 7. Swelling and degradation test for S1, S3, S5, SC1, SC3 and SC5 as labeled in 24 h.

FIG. 8. Equilibrium swelling of SC1, SC3 and SC5 (n=3) in PBS pH 7.4 at 37° C.

FIG. 9. The results of the compression test for SC and S scaffolds, n=5.

FIG. 10A. FTIR spectrum of S5.

FIG. 10B. FTIR spectrum of SC5.

FIG. 11A. Scaffolds weight changes in SBF solution at (top) 3 days and (bottom) 7 days.

FIG. 11B. Scaffolds weight changes in SBF solution at 14 days.

FIG. 12A. Weight changes in SBF solution during 3 days for SC5.

FIG. 12B. Weight changes in SBF solution during 14 days for SC0.

FIG. 13A. SEM images of SC5 before immersing in SBF solution.

FIG. 13B. SEM images of SC5 after 14 days immersing in SBF solution.

FIG. 14. Gravimetric analysis SC0 and SC5 at 7, 15, 30, 45 and 60 days.

FIG. 15. MTT assay of MG63 with different weights of the biomaterials in 3 days.

FIG. 16. MTT assay of MC3TC with different weights of the biomaterials in 7 days.

FIG. 17. Cell proliferation MTT assay for MC3TC after 5 days.

FIG. 18. Cell proliferation MTT assay for MG63 after 5 days.

FIG. 19A. SEM images of scaffold after planting MG63 cells in 5 days; (top) SC5 and (bottom) SC3.

FIG. 19B. SEM images of scaffold after planting MG63 cells in 5 days; (top) SC1 and (bottom) SC0.

FIG. 20A. SEM images of scaffold after planting MC3TC cells in 5 days; (top) SC5 and (bottom) SC3.

FIG. 20B. SEM images of scaffold after planting MC3TC cells in 5 days; (top) SC1 and (bottom) SC0.

FIG. 21. Cross section images of unmodified (S) and after modification (SC) scaffolds.

FIG. 22. Closed percent porosity unmodified Ss and modified SCs scaffolds.

FIG. 23. Connectivity density of unmodified (S) and modified (SC) scaffolds.

FIG. 24. Structural thickness of unmodified (S) and modified (SC) scaffolds.

FIG. 25. Total porosity unmodified (S) and modified (SC) scaffolds.

FIG. 26. Average percent object of the unmodified (S) and modified (SC) scaffolds.

### DETAILED DESCRIPTION

An appropriate and functional regenerative scaffold possesses a three-dimensional structure with three main requirements: i) biological requirements, ii) structural requirements, and iii) mechanical requirements.

Biological requirements: Primary bone cells are cultured in the matrix of a fabricated scaffold; thus, the cells require to adhere and proliferate in the matrix. Moreover, the biocompatibility feature (i.e., nontoxic and nonimmunogenic) is another essential specification of a regenerative implanted scaffold. Biodegradability and bioresorbable properties of the new generation of scaffolds facilitate the bone tissue formation and regeneration process. The resulted products from the degradation of the matrix should be nontoxic and excreted from the body, with no interference with other organs.

Structural requirements: Structural scaffold specifications include the degree of porosity, pore architecture, including size, geometry, orientation, and interconnectivity of pores, and nanotopography are pivotal factors in osteogenic and angiogenic processes leading to bone formation and tissue regeneration. The high degree of porosity provides proper cell growth and migration, nutrient flow, vascularization, and spatial organization that is crucial in bone regenerative tissue engineering. Native bone is composed of cortical (a dense compact shell with porosity 5-10%) and trabecular (porous cancellous core with porosity 70-90%). Some reports have shown that the ideal degree of porosity to mimic natural bone is 60-95%. Although pore sizes ranging from 20-1500 μm have been reported in bone tissue engineering applications, the average pore size ranging from 75 to 100 μm is the minimum requirement to grow bone tissue significantly. The optimal pore size larger than 100 μm is appropriate to grow bone tissue in a bone scaffold, whereas at least 300 μm pores' average size is essential for vascularisation. Interconnected and oriented pores with suitable geometry facilitate cell growth, tissue regeneration, and vascularization. Nanotopography can be a surface structural characteristic of regenerative bone scaffolds. Nanotopography demonstrates nanoscale surface features in the range of 1-100 nm (in some cases a few hundred nanometers). Notably, a landscape at a nanosized scale leads to a dramatically enhanced surface area, eventually resulting in high surface activity as well as the formation of many surface substructures. Nanotopography regulates cell proliferation, differentiation, adhesion, morphology, and migration under the influence of surface roughness.

Mechanical requirements: Mechano-sensitive tissues refer to supporting tissues that are continuously exposed to mechanical force. Bone is one of the mechano-sensitive tissues that is subjected to endure external forces. For this reason, appropriate mechanical properties are a pivotal feature of a fabricated bone scaffold to mimic natural bone. Therefore, a scaffold possesses sufficient mechanical properties is necessary for optimal performance in bone tissue engineering. The optimal values of mechanical strength and modulus of a designed scaffold should be matched to the values of strength (5-10 MPa) and modulus (50-100 MPa) for cancellous bone. The optimum mechanical values of the bone scaffold vary in respect to its anatomical site of the defect and applications in bone tissue engineering.

Various materials have been used to fabricate a bone regenerative tissue scaffold. Ceramics, natural polymers, and synthetic polymers have been employed to fabricate a scaffold for bone tissue engineering. Natural polymers demonstrated biocompatible, biodegradable, appropriate cell adhesion, and osteogenic properties with low mechanical strength. Whereas, synthetic biodegradable polymers, mostly polyesters, exhibited disadvantages such as toxic by-products (acidic degradation), rapid strength degradation in vivo, and poor cell adhesion, and advantages including tunable properties and higher mechanical strength.

Chitosan (CS) is a biocompatible (non-toxic and non-immunogenic), biodegradable, and the second most abundant naturally occurring polymer. Contrary to many synthetic polymers, chitosan possesses a hydrophilic surface that improves cell adhesion and proliferation, while its degradation products are non-toxic. Moreover, chitosan is utilized to fabricate bone scaffolds, providing an osteocompatible matrix to promote cell adhesion and proliferation of bone-forming osteoblast cells and calcium deposition. Studies have reported that modified chitosan scaffolds perform osteoconductivity in vivo to treat bone defects. Chitosan exhibits two main disadvantages: poor mechanical properties and large size swelling expansion. Notably, chitosan performs a large size expansion/swelling as a result of absorbing a lot of water caused by the hydrophilic nature of the biopolymer. Therefore, the pore sizes of chitosan biomaterials are significantly contracted and collapsed with water swelling. It impedes cell proliferation inside pores due to swelling squeezing pores. The scaffolds of this study were designed to solve the disadvantages of chitosan while using the benefits of the excellent advantages of chitosan.

Hydroxylapatite (Ca₁₀(PO₄)₆(OH)₂) (HA) is an important inorganic material, which is noninflammatory, nontoxic, nonimmunogenic, osteointegration, osteoconductive, and biocompatible, utilized in bone tissue engineering. HA, constitutes ˜70% w/w human bone tissue. In addition, HA promotes bioactivity characteristics as well as mechanical properties in bone scaffolds.

Cellulose nanocrystals (CNCs), naturally occurring as sustainable materials, are produced from the hydrolysis of the amorphous parts of native cellulose chains. The remaining residue of the controlled hydrolysis reaction is called CNC nanoparticles, which possess a high degree of crystallinity. Nanosized scale with a high aspect ratio and high crystallinity characteristics introduces unique properties such as a high surface area, rod shape structure, appropriate mechanical properties in materials science. Moreover, CNC nanoparticles offer advantages in biomedical applications including biocompatibility, and nontoxicity. Concerning the aforementioned physicochemical and biological characteristics CNCs. CNC nanoparticles are utilized for drug delivery and tissue engineering applications. In a recent study, a bone scaffold was fabricated by chitosan and alginate along with hydroxyapatite and CNC. The fabricated porous scaffold comprising CNC exhibited cell adhesion and proliferation on the scaffold.

Functional regenerative 3D scaffolds that could meet the above three requirements were engineered using various combinations of chitosan, hydroxylapatite, and cellulose nanocrystals into a bio-nanocomposite which included citric acid as a crosslinker.

Two series of nano-engineered bio-nanocomposites (S and SC) were fabricated, characterized, and evaluated for bone tissue engineering application. The freeze-drying method was employed to fabricate S0, S1, S3 and S5 (S series) scaffolds with three components chitosan, cellulose nanocrystals (CNC) and hydroxyapatite (HA) in different quantities. S0, S1, S3, and S5 were crosslinked using citric acid to fabricate SC0, SC1, SC3, and SC5 counterparts. The reaction of citric acid with the CNC and chitosan as a crosslinker was shown by FTIR spectroscopy. An optimal bio-nanocomposite was SC5 having a maximum amount of CNC and crosslinking bonds. SEM and Micro-CT images of SC5 revealed 90% porosity, long pores with an average width size of greater than 26 micrometers with a cylindrical layered structure. The comparison of XRD patterns, as well as DSC thermograms of bio-nanocomposites and three components, showed that neither a simple blending occurred but also new interactions between three components conducted during the fabrication process. SC5 showed an excellent size expansion swelling, suitable swelling ratio, and swelling kinetic behavior as well as an appropriate degradation. Bioactivity was quantitively measured by using the adsorption value of calcium phosphate in SBF solution. SEM and gravimetry analysis of bio-nanocomposites indicated excellent bioactivity. Compressive modulus was measured for the fabricated scaffolds. CNC value and crosslinking bond improved compressive modulus (about 50 MPa) of bio-nanocomposites. 2D and 3D cell cultures were conducted using two cell lines MG63 human bone osteosarcoma and MC3T3 osteoblast mouse. The cell culture results showed that bio-nanocomposites were nontoxic with cell adhesive characteristics. SEM images of 3D cell-cultured scaffolds revealed that both the bone cell lines properly placed, proliferated, and migrated inside the pores of 3D scaffolds. Thus, SC5 scaffolds were determined osteoconductive and nontoxic biomaterials suitable for bone tissue engineering. This study revealed that the presence of CNC nanoparticles and crosslinking bonds are crucial in the optimal scaffold. CNC nanoparticles introduced a nanotopography pattern on the surface of the chitosan matrix to promote bioactivity tremendously. CNC nanoparticles and crosslinking bonds improved compressive modulus as well as the firm structure of pores with controlled-size expansion swelling in the optimal bio-nanocomposites.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “gene” is defined herein to include a coding region for a protein, peptide or polypeptide

When referring to two nucleotide sequences, one being a regulatory sequence, the term “operably-linked” is defined herein to mean that the two sequences are associated in a manner that allows the regulatory sequence to affect expression of the other nucleotide sequence. It is not required that the operably linked sequences be directly adjacent to one another with no intervening sequence(s).

The term “regulatory sequence” is defined herein as including promoters, enhancers and other expression control elements such as polyadenylation sequences, matrix attachment sites, insulator regions for expression of multiple genes on a single construct, ribosome entry/attachment sites, introns that are able to enhance expression, and silencers. Promoters may be cell specific or tissue specific to facilitate expression in a desired target.

The term “vector” is used interchangeably with the terms “construct”, “DNA construct”, “genetic construct”, and “polynucleotide cassette” to denote synthetic nucleotide sequences used for manipulation of genetic material, including but not limited to cloning, subcloning, sequencing, or introduction of exogenous genetic material into cells, tissues or organisms, such as animals. It is understood by one skilled in the art that vectors may contain synthetic DNA sequences, naturally occurring DNA sequences, or both. The vectors of the present invention are transposon-based vectors as described herein.

### Embodiments of the Technology.

This disclosure provides a bio-nanocomposite comprising chitosan (CS), cellulose nanocrystals (CNC) and apatite (XA) wherein the mass ratio CS:CNC is about 0.5:1 to about 10:1, CS is crosslinked to CNC via a linker to form a polymer matrix (PM), and XA is uniformly dispersed in the polymer matrix wherein the mass ratio of XA:PM is about 1:1 to about 1.5:1.

In some embodiments, the mass ratio CS:CNC is about 1:1 to about 5:1. In some embodiments, the mass ratio CS:CNC is about 0.5:0, about 1:0, about 0.5:1, about 1:1, about 2:1, about 3:1, about 4:1, about 5:1, about 6:1, about 7:1, about 8:1, or about 9:1. In some embodiments, the weight percent of CNC in the bio-nanocomposite is about 5%, about 10%, about 15%, about 20%, about 25%, about 30%, or about 35%. In some embodiments, the bio-nanocomposite does not include any added CNC.

In some embodiments, the mass ratio XA:PM is about 1.1:1 to about 1.2:1. In some embodiments, the mass ratio XA:PM is about 1:1, about 1.1:1, about 1.2:1, about 1.3:1, or about 1.4:1. In some embodiments, XA is sintered hydroxylapatite (HA) (also referred to as hydroxyapatite). In some embodiments, XA comprises sintered hydroxylapatite (HA). In some embodiments, XA comprises HA.

In some embodiments, the linker comprises an organic acid. In some embodiments the organic acid has two or more carboxyl groups. In some embodiments, the organic acid has two, three or four carboxyl groups. In various embodiments, the bio-nanocomposite comprises the organic acid in an % w/w amount of about 1%, about 2%, about 3%, about 4%, about 5%, or about 6% w/w.

In some embodiments, the organic acid is citric acid wherein the carboxyl groups on citric acid are covalently linked to the hydroxyl moieties on CNC via an ester bond and are covalently linked to the hydroxyl moieties on chitosan via an ester bond and/or the carboxyl groups on citric acid are covalently linked to the amine moieties on chitosan via an amide bond.

In some embodiments, CNC is an oxidized CNC comprising added carboxyl groups wherein the carboxyl groups are covalently linked to the hydroxyl moieties on chitosan via an ester bond, the carboxyl groups are covalently linked to the amine moieties on chitosan via an amide bond, or a combination thereof.

In some embodiments, the bio-nanocomposite comprises interconnected pores. In some embodiments, the bio-nanocomposite has about 60% to about 90% porosity. In some embodiments, the porosity is about 55%, about 65%, about 70%, about 75%, about 80%, about 85%, or about 95%. In some embodiments, the interconnected pores have an average pore diameter of about 10 micrometers to about 30 micrometers.

In some embodiments, the linker is citric acid, the apatite is sintered hydroxylapatite (HA), the mass ratio CS:CNC is about 1:1 to about 5:1, and the mass ratio HA:PM is about 1.1:1 to about 1.2:1. In some embodiments, the bio-nanocomposite comprises interconnected pores and about 90% porosity, wherein the interconnected pores have an average pore diameter of about 25 micrometers to about 30 micrometers. In some embodiments, the average pore diameter is about 5 micrometers to about 50 micrometers. In some embodiments, bio-nanocomposite further comprises bone cells. In some embodiments the bio-nanocomposite further comprises bone cells or cartilage cells and one or more growth factors (or cells or genes expressing the one or more growth factors).

In some embodiments, the bio-nanocomposite has a compression modulus at least two-times greater than a corresponding bio-nanocomposite that is not crosslinked. In some embodiments, the compression modulus is at least four-times or at least eight-times greater than a corresponding bio-nanocomposite that is not crosslinked. In some embodiments, the bio-nanocomposite has a weight loss of about 5% or less, about 10% or less, or about 25% or less in phosphate buffered saline (PBS) at pH 7.4 at about 37° C. after 7 days, 15 days, 30 days, or 60 days, or up to 90 days.

Also, this disclosure provides a method for forming a bio-nanocomposite artificial matrix for bone or cartilage comprising:

a) contacting chitosan (CS), cellulose nanocrystals (CNC), sintered hydroxylapatite (HA) and an aqueous solution at about pH 5 to about pH6 to form a mixture;

b) freezing the mixture in a cast and lyophilizing the frozen mixture to form a scaffold;

c) contacting the scaffold, citric acid (CA), and an alcohol; and

d) removing excess citric acid and the alcohol;

wherein the mass ratio of CS:CNC is about 0.5:1 to about 10:1, CS is crosslinked to CNC via citric acid to form a polymer matrix (PM), and the mass ratio of HA:PM is about 1:1 to about 1.5:1; and

wherein HA is uniformly dispersed in the polymer matrix and the artificial matrix is thereby formed.

In some embodiments, the artificial matrix comprises about 0.2% w/w to about 10% w/w of the citric acid (CA). In various embodiments, the artificial matrix comprises about 2% w/w to about 6% w/w of the CA, or about 4% of the CA. In some embodiments, the artificial bone matrix or artificial cartilage matrix comprises interconnected pores and about 90% porosity, wherein the interconnected pores have an average pore diameter of about 25 micrometers to about 30 micrometers.

In some embodiments, the method further comprises contacting the artificial matrix, bone cells or cartilage cells, and optionally one or more growth factors, under suitable physiological conditions for bone or cartilage regeneration. In some embodiments, suitable physiological conditions comprise calcium ions, phosphate ions, calcium phosphate, or a combination thereof. In some embodiments, suitable physiological conditions comprise a cell culture medium or minimum essential medium (MEM) such as Eagle's MEM, alpha-MEM, Dulbecco's MEM, Glasgow's MEM, blood plasma, body fluid, or simulated body fluid (SBF) having an ion concentration comparable to that of human blood plasma.

Additionally, this disclosure provides a method for regenerating bone tissue or cartilage tissue comprising contacting the bio-nanocomposite according to the disclosed bio-nanocomposite disclosed herein and bone cells or cartilage cells under suitable physiological conditions wherein bone tissue or cartilage tissue is thereby regenerated. In some embodiments, contacting is performed in a subject in need of bone regeneration or cartilage regeneration. In various embodiments, the bio-nanocomposite or artificial matrix is osteoconductive for proliferation of bone cells or cartilage cells.

### Results and Discussion.

Chitosan was utilized as the matrix of the bio-nanocomposites. The formulations were composed of various ratios of chitosan and CNC as the organic part of the scaffold and the amount of HA is almost 1.2-fold (54%) of total organic materials. The organic components adsorbed HA with a maximum amount (54%) of total organic components (chitosan and CNC). Using more than 54% of HA caused to precipitate the extra HA in the reaction mixture.

Two types of hydroxylapatite sintered (S) and unsintered (R) hydroxylapatites were separately examined. In general, sintered hydroxylapatite with 5.6 μm particle size possesses a high shear strength compared to unsintered one. A study was shown that the sintering process improved mechanical properties in HA materials. Shrinkage was notably observed in the freeze-dried biomaterials with unsintered HA. Therefore, scaffolds with unsintered HA were determined not to be suitable for fabricating the bone scaffold with the freeze-drying method in this project.

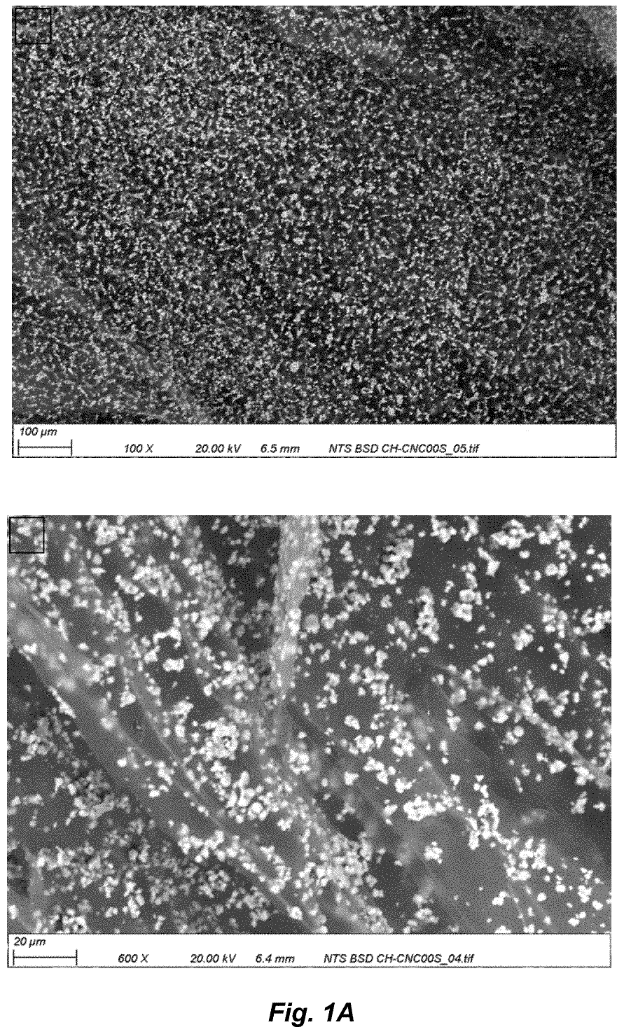

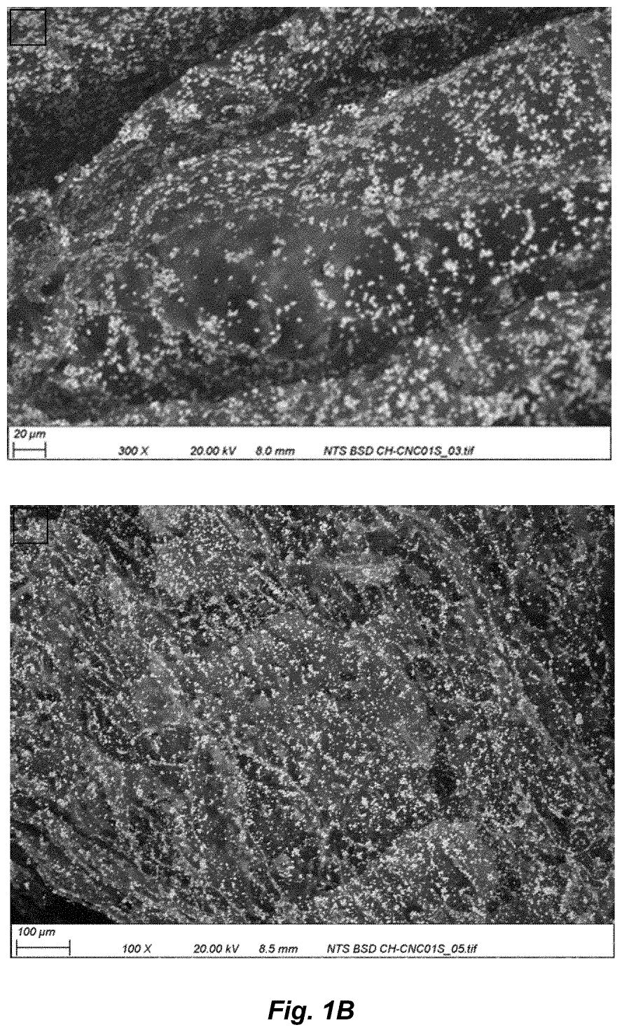

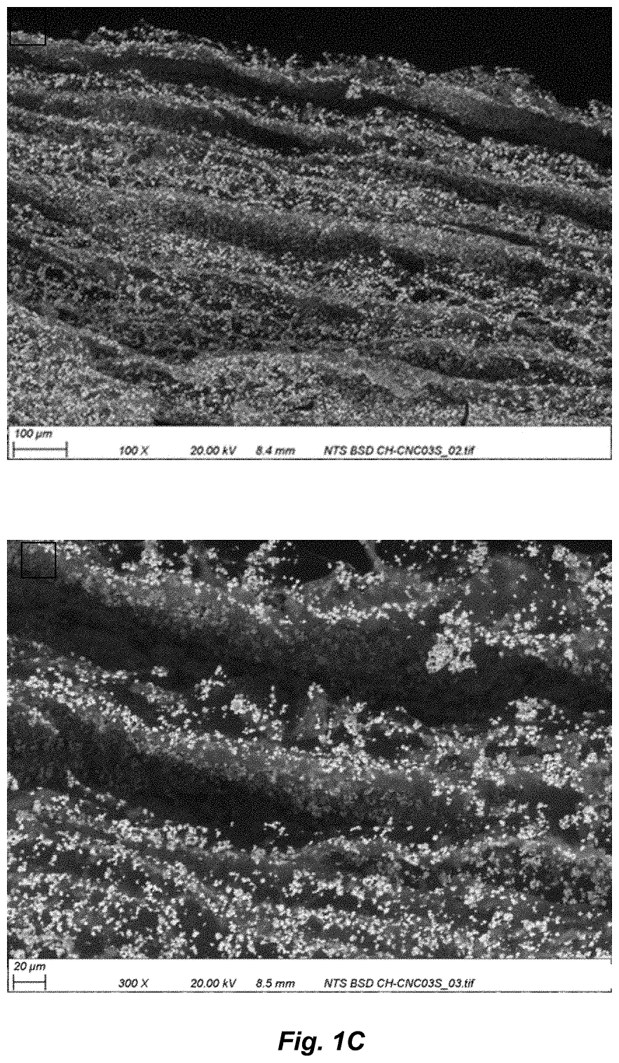

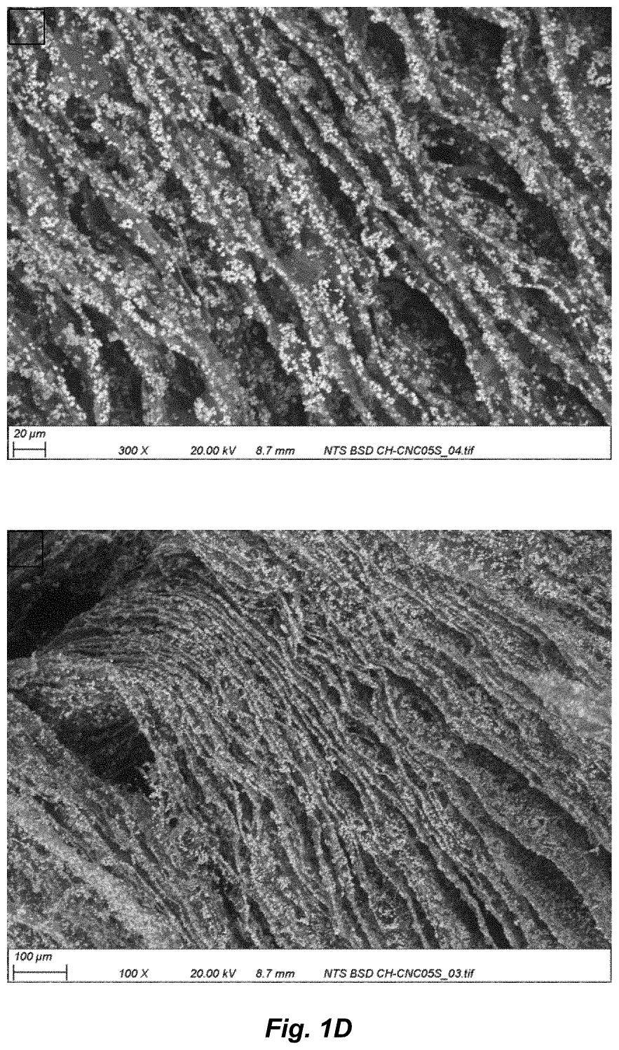

SEM images of the cross-section of the bio-nanocomposite S series are shown in FIG. 1. S0 (FIG. 1A) bio-nanocomposite, which contains chitosan and HA without CNC (control). The number of micro-scale pores of this bio-nanocomposite is very low, and the pore size is mostly in nanoscale. The micro-scale pore width is about 12.49 μm. HA particles were adsorbed on chitosan/CNC fibers and observed as white spots in the SEM images. FIG. 1B is shown SEM images of S1. The pore sizes in S1 are larger compared to S0. The average of the pore width in this formulation is 19.75 μm. The SEM images of the bio-nanocomposite S3 are indicated in FIG. 1C. The images show long pores with increasing porosity compared to S1. The average pore width for this bio-nanocomposite is ˜23 μm. Blind and interconnected pores are observed in S3 sample. CNC improved the interconnectivity of pores in the structure of S3, but this bio-nanocomposite still contained some blind (capped) pores. HA was adsorbed on the materials more uniformly compared to S1. SEM images of S5 (FIG. 1D) bio-nanocomposite remarkably exhibited highly porous texture with long and interconnected pores. The average width of pores in the formulation was 26 μm with uniform distribution in the entire scaffold.

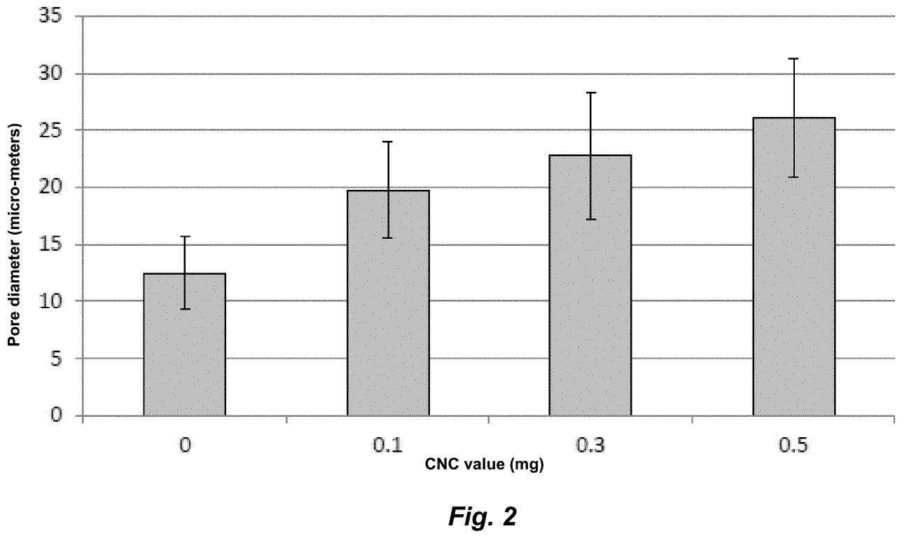

The SEM images of S series bio-nanocomposites showed that most of the prepared scaffolds to be porous biomaterials with micro-pore scales and uniform HA dispersion on the organic components. The increase in the amount of CNC was remarkably associated with enhancing the porosity of the bio-nanocomposites. Increasing CNC content in bio-nanocomposites enhanced pore diameters, as shown in FIG. 2 (P=0.00045895, one-way ANOVA). Moreover, an increasing amount of CNC enlarged the length size of the pores.

Overall, the S5 sample, with a maximum amount of CNC, exhibited larger and uniform pores with a high degree of porosity. The S5 sample exhibited long cylindrical pores with a multilayered 3D interconnected structure and symmetrical distributions of pores that are expected to be very suitable for the fabrication of a bone mimicking scaffolds. Interconnection in the porous materials is an important factor to make the scaffold osteoconductive. The calculation of total porosity based on total volume changes during freeze-drying and volume of initial materials showed that the total porosity is above 90% for S5. The pore diameters of these three scaffolds containing CNC are ranging about 20-25 μm. The 20-25 μm width along with the large length and depth of the pores provided sufficient spaces for cell proliferation and vascularization. Therefore, it makes them a suitable candidate for bone tissue engineering purposes.

The results of EDS analysis of scaffolds showed the presence of calcium (Ca) and phosphorus (P) elements that originated from the inorganic HA component. These results showed that HA must have contributed to all the scaffold structures. The SEM images also clearly indicated HA particles attached to the organic fibers. The EDS data for the CNC showed that the content of the element including carbon and oxygen from cellulose, also sulfur and sodium from the remaining sodium salt of sulfate ester from the hydrolysis preparation process used during the production of CNC. It seems sulfate ester bonds were hydrolyzed during the fabrication of the scaffolds. Hence sulfur and sodium content in the scaffolds are zero.

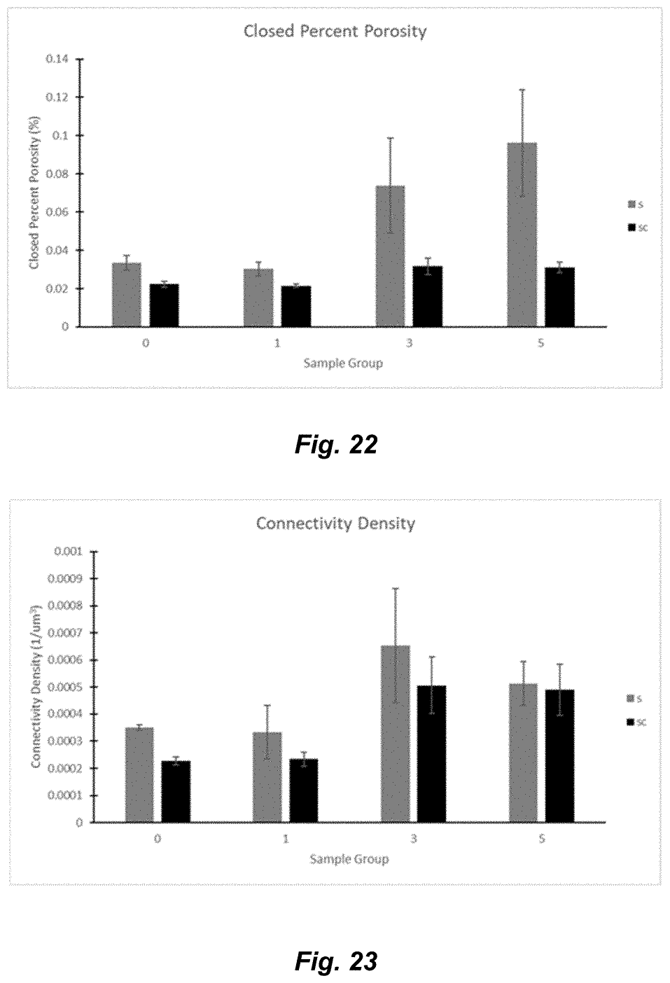

The Micro-CT images confirmed the cylindrical structure of the pores, of bio-nanocomposites of S5. HA was more uniformly dispersed in the structure of the S5 bio-nanocomposite. The calculated results of Micro-CT of S5 showed that S5 contains 65.44% open pores and less than 1.14% closed pores (see FIG. 22).

Chitosan exhibits relatively a large amorphous hole in the XRD pattern. The amorphous hole of the pure chitosan has been disappeared while it involved in the bio-nanocomposite structure. The XRD pattern of S5 indicated the presence of all the sharp peaks, corresponding to the crystalline parts of HA, CNC, and chitosan with no amorphous hole. Consequently, the presence of CNC and HA in the bio-nanocomposite induced ordered chitosan chain structure to form S5 bio-nanocomposite. This result agrees well with SEM and Micro-CT image specifications. The ordered structure led to the creation of interconnected pores in the scaffold, suitable for bone cell implantation and growth.

Two endothermic sharp peaks at 155 and 190° C. showed in the chitosan's DSC thermogram. CNC thermogram indicated a broad peak at 90° C. that is responsible for water content evaporation Whereas the chitosan endothermic peaks disappeared in S5 bio-nanocomposite. It showed a broad endothermic peak starting at 160° C. Overall, the XRD and DSC interpretations implied that the fabricated bio-nanocomposite is not a simple blend of three components. The results indicated the formation of new structures in this mixture due to the interaction of its components.

An appropriate bone scaffold offers not to be expanded significantly in an aqueous or buffer media. Pores will be squeezed in case of high expansion swelling of the scaffold, and the cells will be taken out of the scaffolds if that is the case. A large swelling expansion was observed in the case of the chitosan matrix of the S series.

In addition, retained 3D structure in an aqueous media is crucial for bone scaffolds. S series were dissociated and dispersed in an aqueous medium with different rates. S1 nanocomposite was dissociated and dispersed in 10 minutes with no swelling. The S3 composite, swelled very rapidly, where its size doubled in less than 10 minutes. The S3 scaffold was dissociated after 15 minutes and dispersed after 24 hours. S5 was expanded double during the swelling, in 15 minutes. The S5 was dissociated after 30 minutes and dispersed after 48 hours.

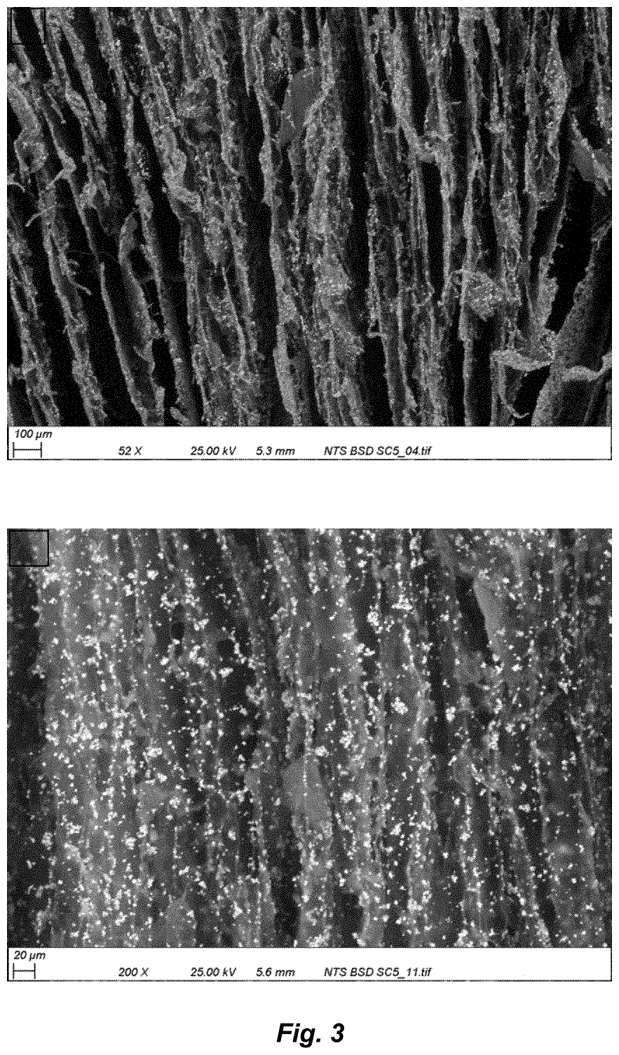

Thus, observed results on the S series scaffolds showed that the scaffolds had to be modified to control their swelling, dissociation and improve their mechanical properties. Citric acid is nontoxic and a multifunctional carboxylic acid that is a crosslinker between chitosan chains and, chitosan and CNC particles. The S scaffolds were modified by adding 4% citric acid to the formulations followed by a thermal process to occur crosslinking chemical reactions between the biopolymers. The new crosslinked series called SCs series (SC1, SC3, and SC5). SEM images of SC1, SC3, and SC5 showed the pore structures and sizes maintain very well (FIG. 3).

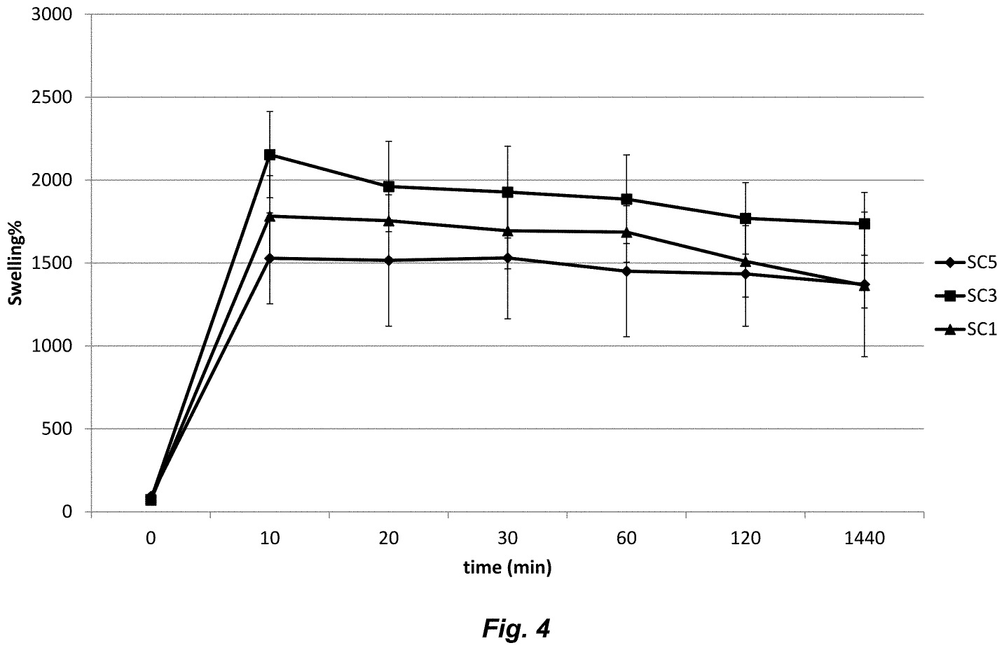

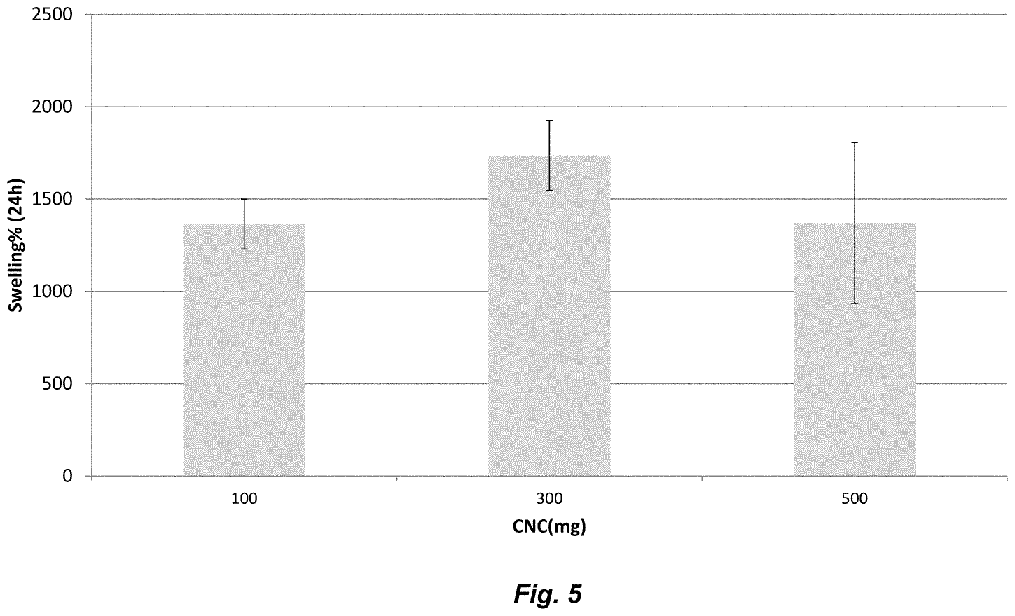

A swelling study for SCs scaffolds was carried out in the water. As shown in FIG. 4, the swelling of all SC samples followed the same pattern; it was fast in the first 10 minutes and reached a plateau after. The rates of swelling were very fast because of the high porosity of the developed materials. The equilibrium swelling for SC samples was also high; approximately 1500% for the three SC samples (FIG. 5).

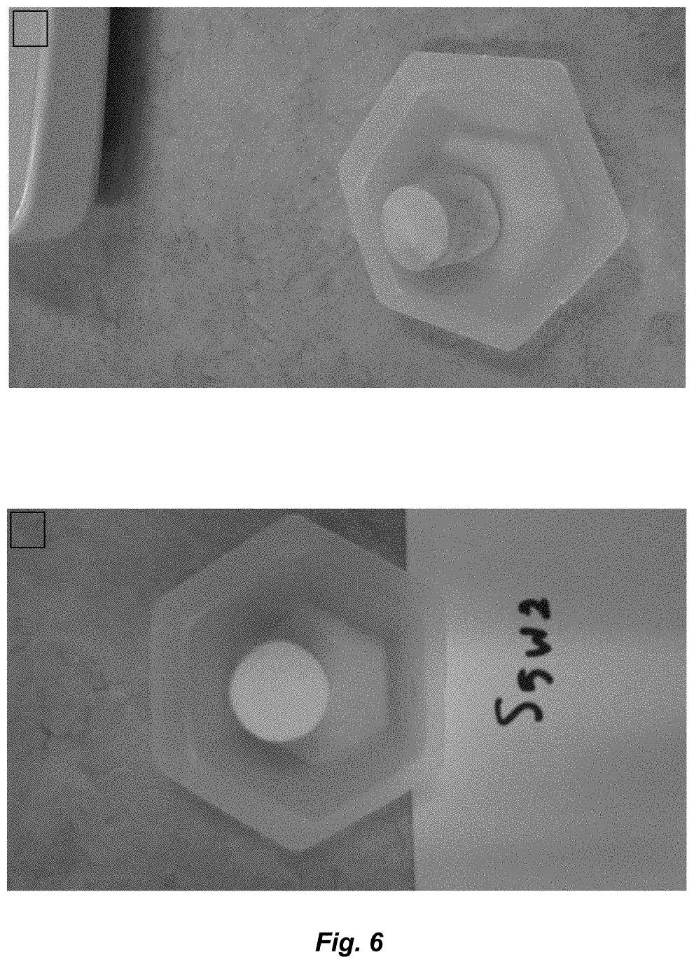

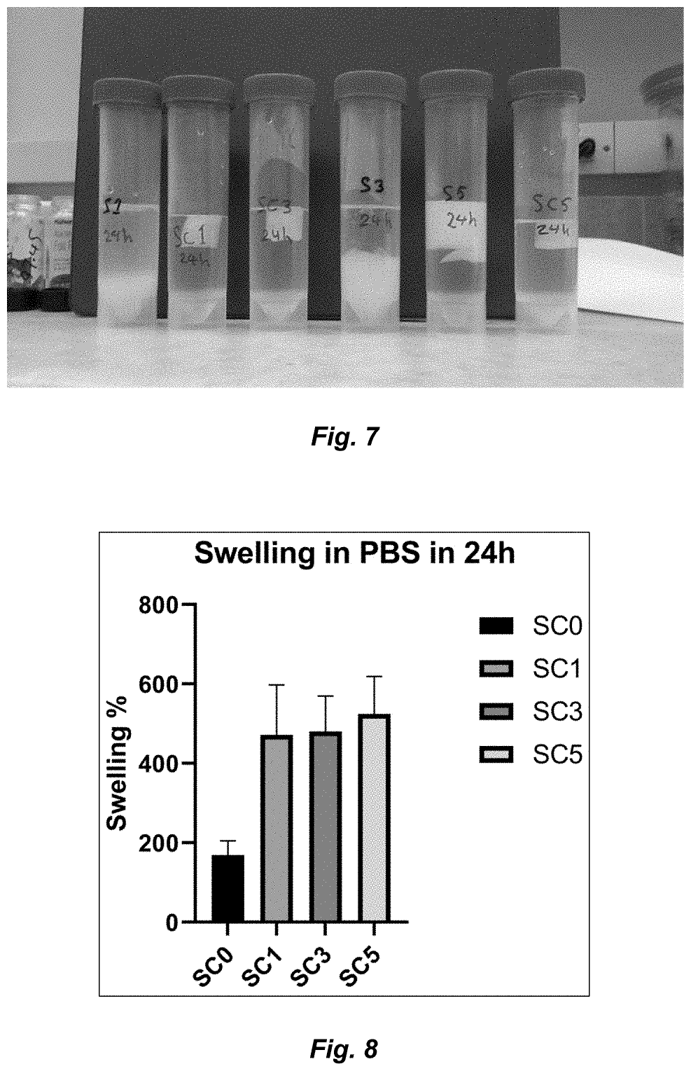

The initial sizes of SC5 and S5 scaffolds (before swelling) are shown in FIG. 6, top panel, and bottom panel, respectively, while the changes in their shape and volume during the swelling (24 h) experiment are shown in FIG. 7. This is also shown for S1, S3, S5, SC1, SC3 and SC5 samples in FIG. 7. No size change was observed in SC1, SC3, and SC5 during their swelling as shown in FIG. 7. Whereas S1, S3, and S5 were dissociated in water, the texture of the scaffolds was degraded in water as shown in FIG. 7. SC1 and SC3 were softened and retained their shapes with swelling, but SC5 structure was maintained firm with no changes in its shape after swelling. The high content of CNC and highly crosslinked texture of the bio-nanocomposites by citric acid elucidated the firmed structure of SC5 and retained shapes of the SC series of bio-nanocomposites.

Equilibrium swelling was measured in PBS at 37° C. Results are exhibited in FIG. 8. The swelling in PBS was less than water swelling. SC0 showed less than 200% swelling in PBS. The swelling of SC1, SC3, and SC5 was more than 400%. Statistical analysis showed that the addition of CNC significantly enhances the swelling ratio in PBS. However, increasing the content of CNC did not affect swelling further.

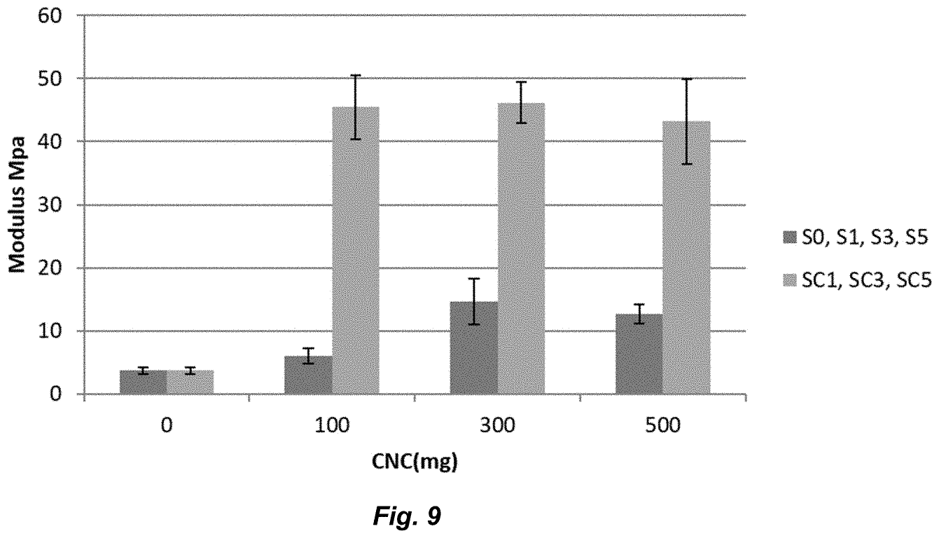

Further studies showed that the compressive strength of the scaffolds was remarkably promoted by increasing the amount of CNC and modification with citric acid. The compressive modulus of S3 and S5 is about 3 to 4 times more than S0 (without CNC). CNC nanoparticles remarkably improved the compressive modulus of the bio-nanocomposites. The compressive modulus of SCs series increased about 4 to 9 folds compared to the unmodified counterpart scaffolds of S series. Although SC1, SC3, and SC5 scaffolds exhibited similar modulus values, with no significant difference. FIG. 9 shows the results of the compression test.

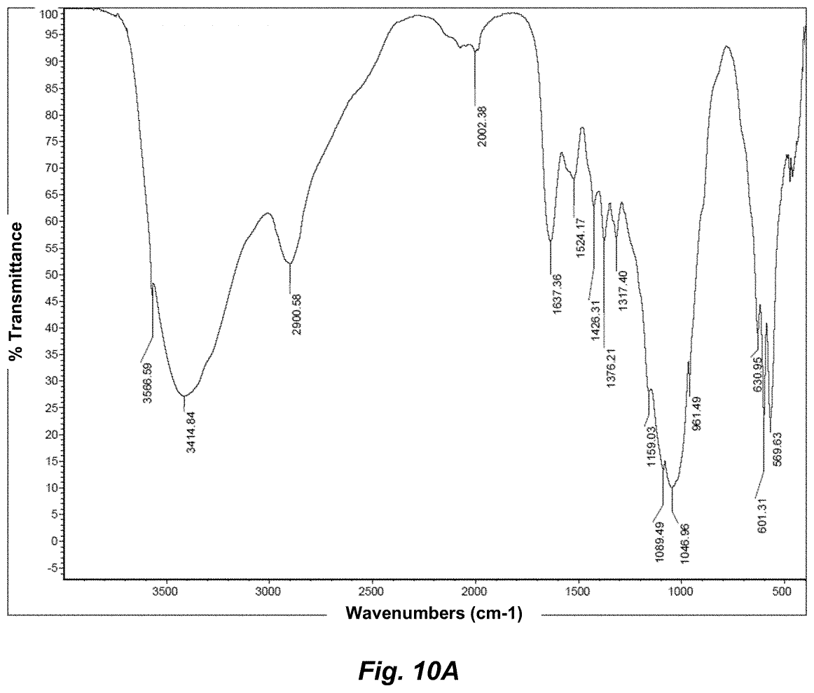

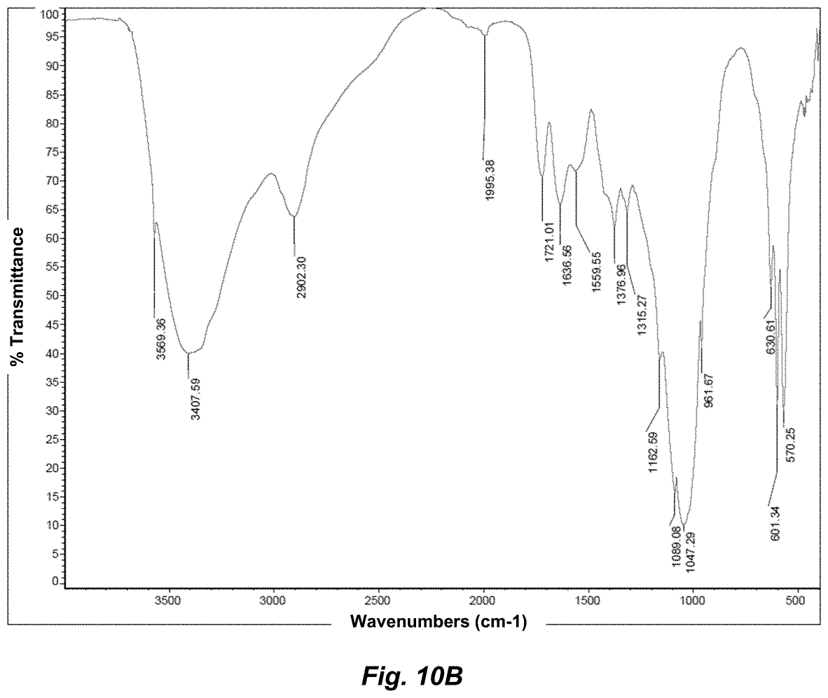

Chemical characterization of SC series was carried out by FTIR (FIG. 10). SC5 showed an extra peak at 1721 cm′, compared to counterpart scaffold S5. This peak is responsible for the carbonyl stretching of carboxylic acid or ester groups. SC3 exhibited a similar extra peak at 1724 cm⁻¹. SC1 indicated an extra peak at 1721 cm′. Ester crosslinked bonds formed from the reaction of citric acid with chitosan chains and CNC, and chitosan chains.

TGA thermograms exhibited excellent thermal stability up to 200° C. (Table 1). The decomposition temperature of the sample at the point of 5% weight loss, defined as T5%. T5% for chitosan and CNC were 86 at 105° C., respectively. The bio-nanocomposites S1, S3, S5, SC1, SC3, and SC5 showed similar thermal stability, but a higher value of T5% compared to starting organic materials. Char yields (the remaining material after decomposition at a certain temperature) of bio-nanocomposites indicated weight loss of each was approximately 5% (at 200° C.) that was attributed to the evaporation of water content of the biomaterials. Chitosan showed weight loss of about 8% at 200° C. likely, due to the evaporation of water content and decomposition of chitosan.

TABLE 1
TGA results for bio-nanocomposites and starting materials.

| | | T5% | Char yield % |
|---|---|---|---|
| Number | Scaffold | (° C.) | at 200° C. |
| 1 | S1 | >200 | 97.3 |
| 2 | S3 | 192 | 94.2 |
| 3 | S5 | >200 | 95.2 |
| 4 | SC1 | 186 | 94.2 |
| 5 | SC3 | 186 | 94.2 |
| 6 | SC5 | >200 | 95.4 |
| 7 | Chitosan | 86 | 91.4 |
| 9 | CNC | 105 | 94.5 |
| 10 | HA | >200 | 99.9 |

The bioactivity of a bone scaffold is characterized by the formation of apatite on its surface when the scaffold is in contact with the physiological environment. It is an essential requirement in artificial bone related biomaterials. This calcium phosphate formation on the scaffold can be evaluated in vitro by incubation of the scaffold in simulated body fluids (SBF)s. Ionic concentrations in SBF are close to human blood plasma ionic concentrations. To mimic the body condition in this study, the scaffolds were immersed in SBF at pH 7.4 and 37° C.

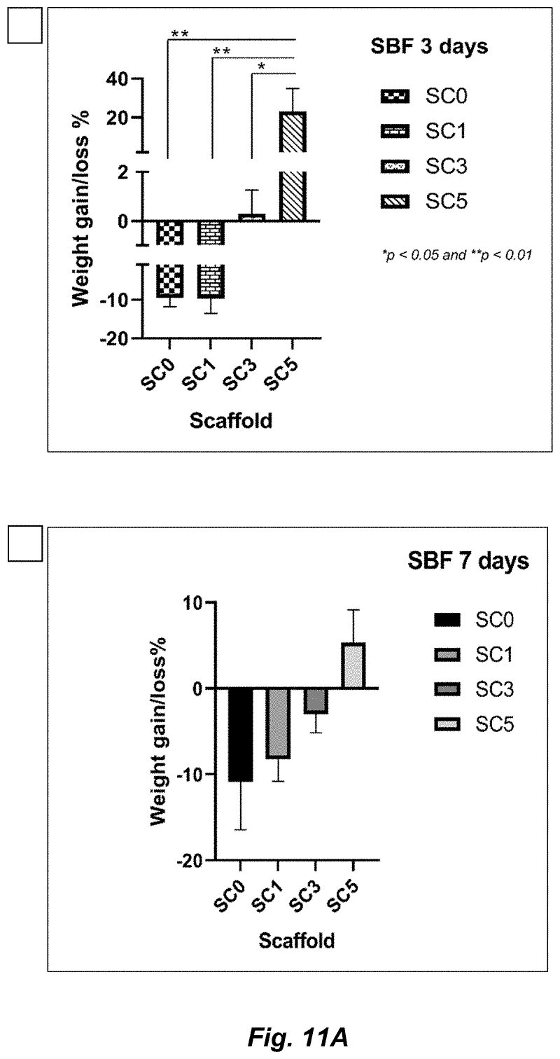

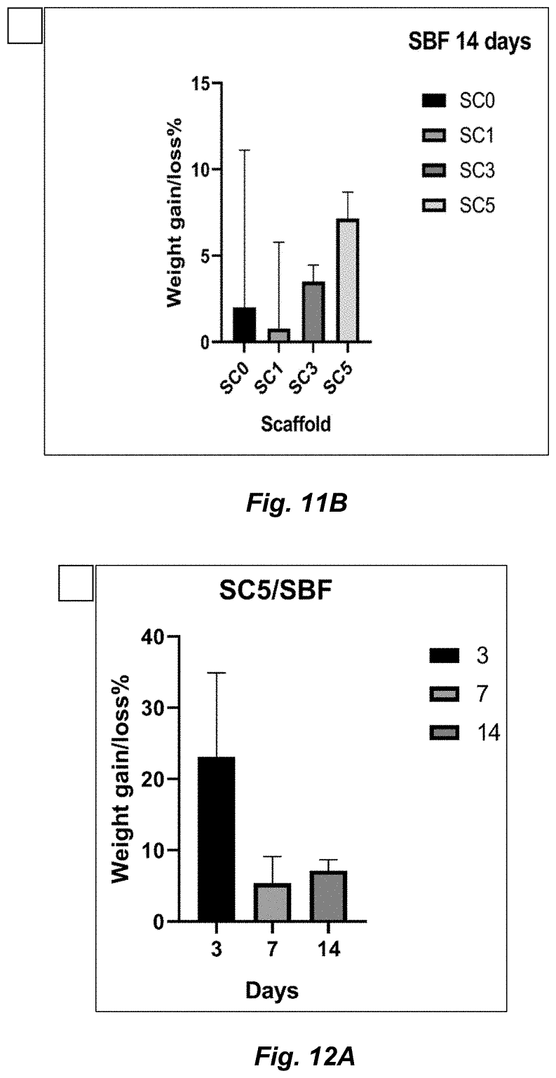

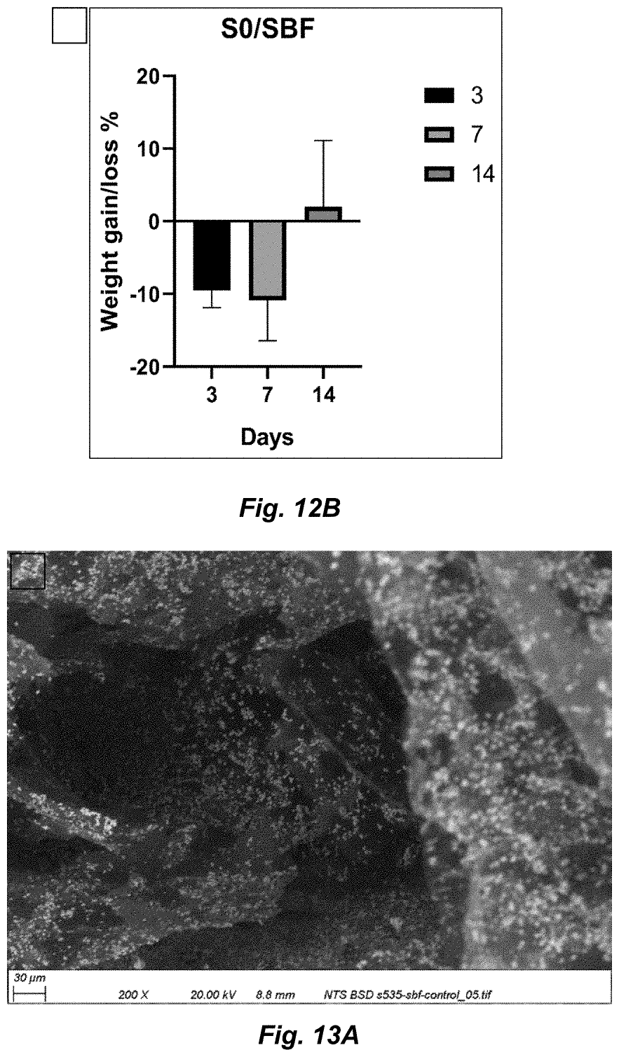

Deposition and dissolution phenomena occurred in this process due to the meta-stable SBF solution. FIG. 11 exhibits bioactivity of the scaffolds in 3, 7 and 14 days. CNC remarkably attributes in calcium phosphate adsorption as shown in FIG. 12. CNC nanoparticles deposited on the chitosan fibers introduced nanotopography on the bio-nanocomposites. The CNC nanoscale landscape on the SCs matrixes elucidates high adsorption of calcium phosphate from SBF due to high surface area to the mass ratio of CNC.

SC5 exhibited a maximum amount of weight gain in 3 days of immersing in SBF solution. The correlation between SC0 to SC5 for deposition of solid material was very significant (p<0.01). It appears that the high content and surface area of CNC contributed to the adsorption of more calcium phosphate on SC5. SC0 and SC1 showed a decreased weight (about 10%) in the first 3 days. But their weights were increased after 7 days. This could indicate the balance between the two opposite phenomena, weight gain due to calcium phosphate deposition and weight loss because of the scaffold degradation. All scaffolds gained weight after 14 days (FIG. 11). Degradation results agree well with this observation.

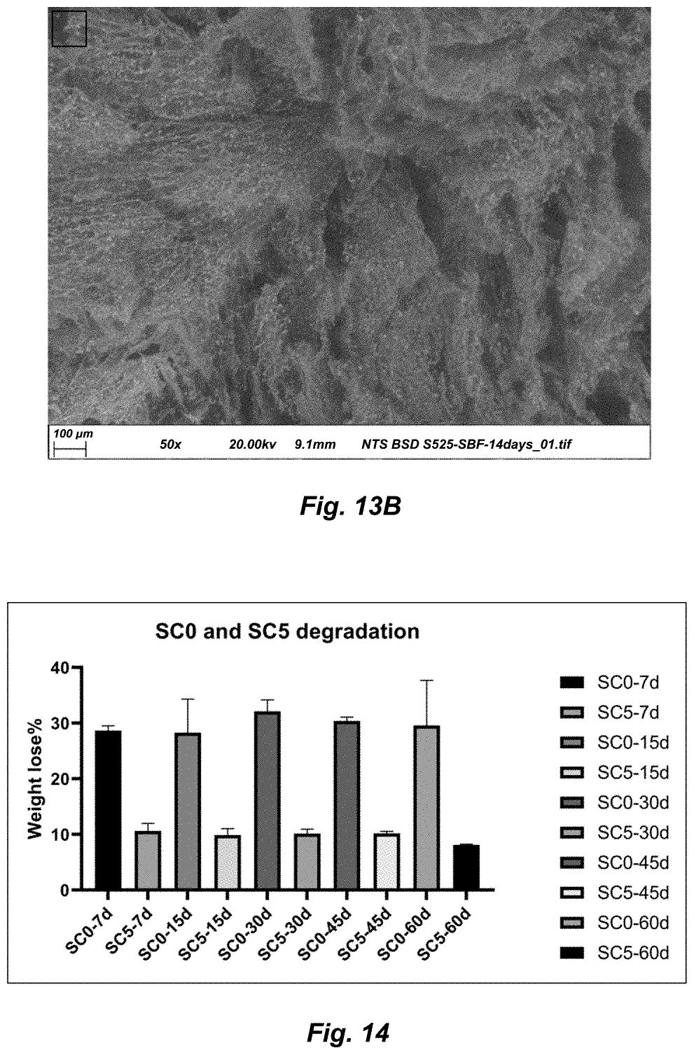

SEM images indicate that calcium phosphate was adsorbed on the surface of the scaffolds as shown in FIG. 13. Thus, CNC plays an important role in the bioactivity of the scaffolds. Increasing CNC content remarkably enhanced the formation of the apatite materials on the surface of the scaffold.

One of the important properties in bone tissue engineering is biodegradation. The scaffolds should be biodegraded and replaced with bone tissue when they are planted in the body. To mimic the body conditions, PBS buffer at pH 7.4 as 37° C. was used to study the degradation of the SC0 and SC5; with time points ranging from 7 to 60 days. Gravimetric analysis and SEM were used to evaluate the structural changes due to the degradation of the scaffolds. The gravimetric analysis results are shown in FIG. 14 for 7, 15, 30, 45 and 60 days.

SC0 indicted about 30% weight loss with a very soft texture after soaking in PBS. On the other hand, SC5 lost approximately 10% of its initial weight. Degradation was slower in SC5 due to more firm macroscopic structures and more stable pore structures. The structure of SC0 were collapsed after a few days in PBS at 37° C., but the structure of the SC5 collapsed in about 30 days in PBS at 37° C. One Way ANOVA with Tukey post-test showed a significant difference (P<0.0001) between the weight loss for SC0 and SC5 at the equivalent time points. SEM images of SC0 and SC5 at 30 days showed that the pore structures were maintained during the degradation process, although the macroscopic structures of the scaffolds were softened after 30 days. Most likely the ester crosslinks were broken during the degradation of the scaffolds.

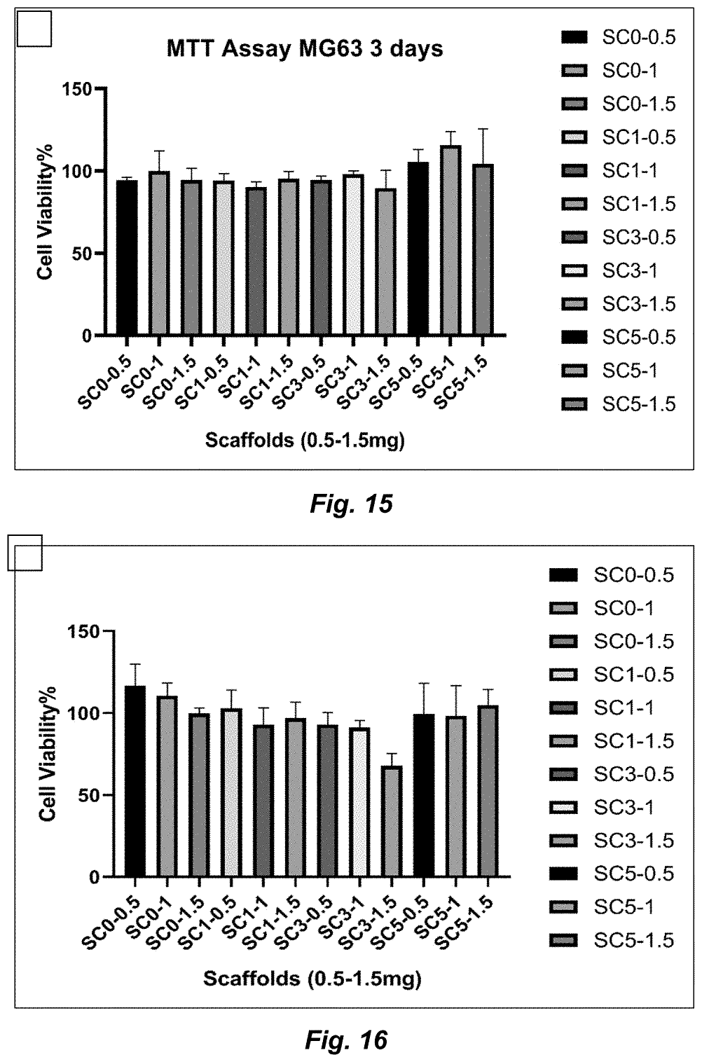

Cytotoxicity MTT assay in 2D culture was conducted with two cell lines. MG63 human bone osteosarcoma and MC3T3 osteoblast mouse both are adherent cell lines were utilized for MTT assay 2D culture with suspending scaffolds in the media of each cell line. The cells were placed at the bottom of each well for 2D culture. The MTT assay (3 days) results for MG63 is shown in FIG. 15 for the various masses of the bio-nanocomposite ranging between 0.5-1.5 mg. Cell viability is more than 80% of the control. The results of MTT assay of MC3TC with different weights of the biomaterials 7 days show in FIG. 16. The cell viability is very high as shown in FIG. 16. Cell viabilities in SC0 and SC5 are more than 100%. Therefore, nontoxic behavior was observed for all scaffolds.

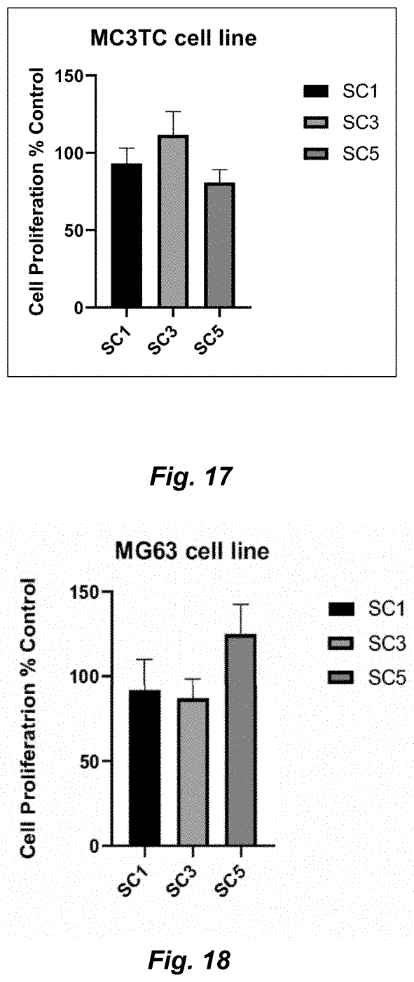

To assess osteoconductive behavior and cell migration, and proliferation in the scaffolds 3D cell culture experiments were performed to mimic the real structure of the scaffold upon use. Cytotoxicity by MTT assay in 3D culture was carried out with a similar protocol of 2D cytotoxicity, but instead of putting the cells on the bottom of each well, the cells were planted on the surface of the scaffolds for 5 days for 3D culture using both cell lines. The results are shown in FIG. 17 for MC3TC proliferation. SC0 is considered as control.

All scaffolds SC1-5 showed cell viability of more than 80% (80% for SC5 and more than 100% for SC3). SC0 (control) 3D structure was collapsed and changed during the cell culture experiments, due to the soft texture of the scaffold. But SC1-5 structures were maintained very well. Cell viability results for MG63 are exhibited in FIG. 18. Cell viability of SC5 was more than 100% as shown in FIG. 18. It seems CNC enhances cell proliferation in MG63 human bone cell line.

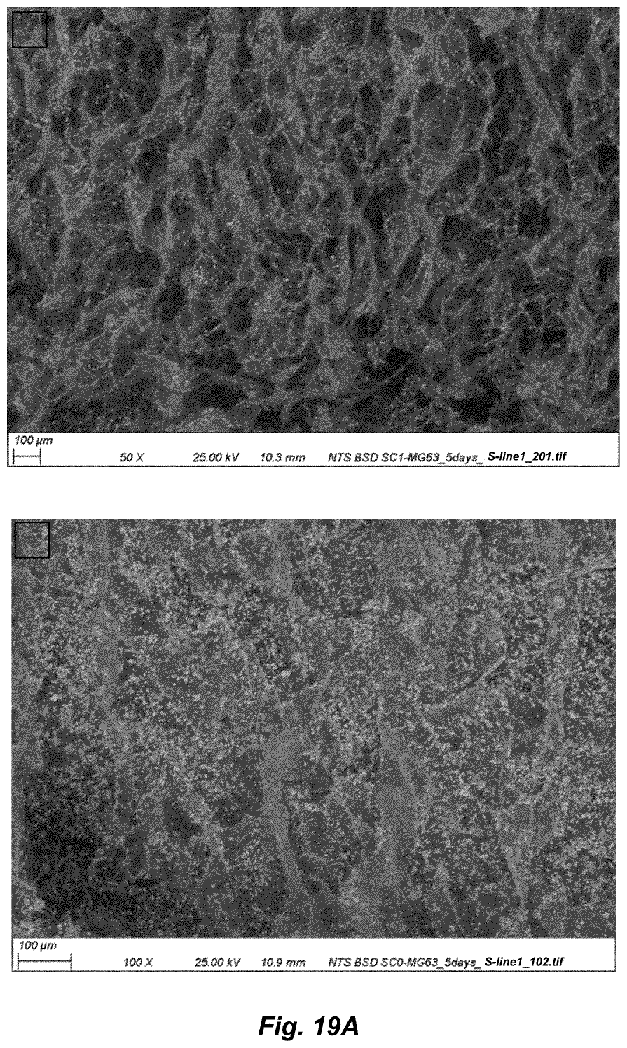

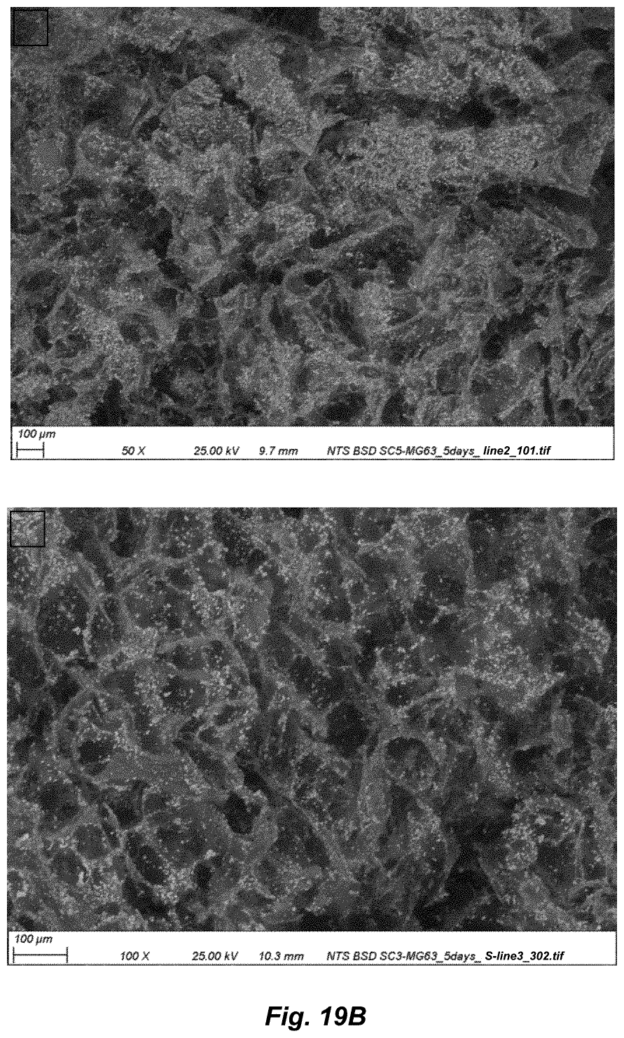

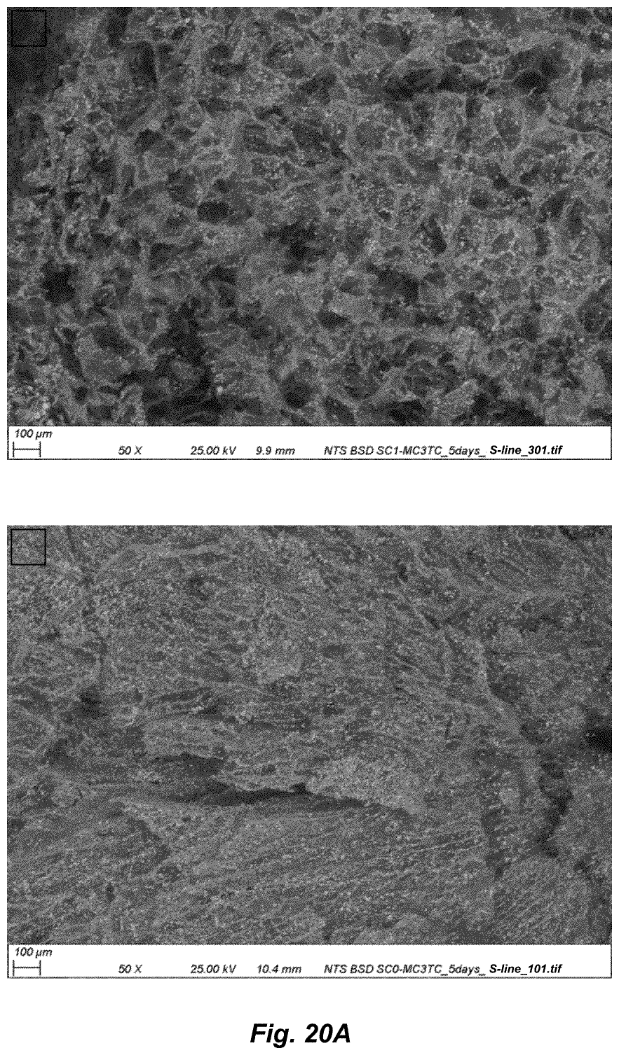

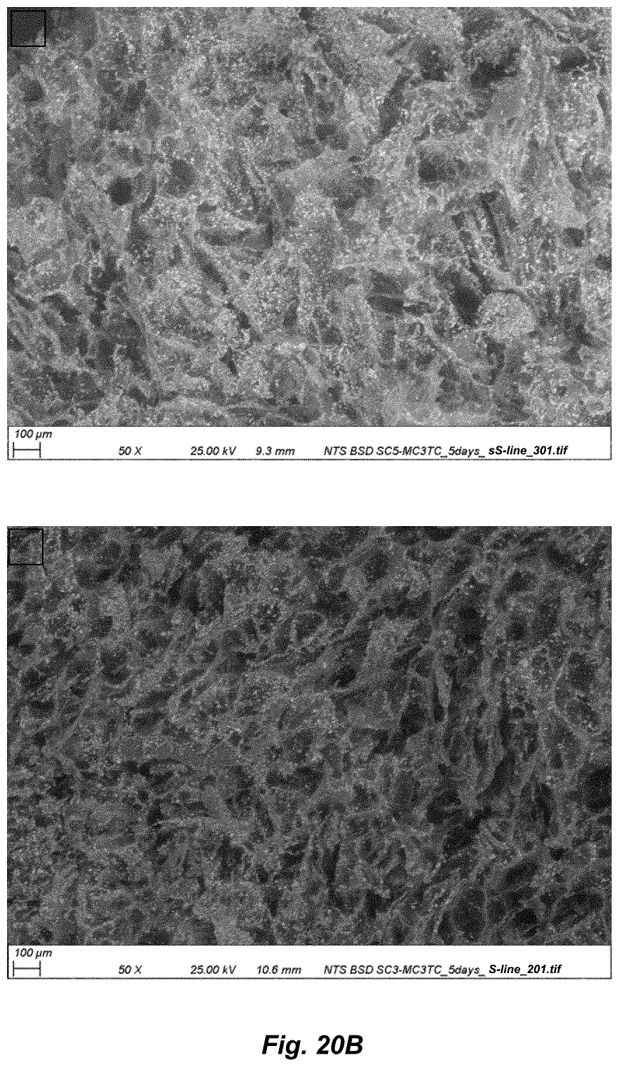

Cell morphology was evaluated by SEM. SEM images were taken from different slices of the cross-section of the scaffolds after fixing the cells in the scaffolds resulted from 3D cell cultures. This study was done for both cell lines. The pores in the treated SC0 scaffolds for both cell lines were destroyed as shown in FIG. 19 and FIG. 20. The pores SC1, SC3, and SC5 were filled with the cells (Mg63 and MC3TC). It implied cells were appropriately accommodated and proliferated in the pores of the bio-nanocomposites. The cells were observed in the lower slices of the scaffold far from the top of the SC5 scaffold where cells were planted on the scaffold. The results showed the cells migrated through the scaffold successfully. Therefore, SC5 scaffolds were determined osteoconductive and nontoxic biomaterials suitable for bone tissue engineering.

Nano-engineered bio-nanocomposite SC5 scaffold is the optimal bio-nanocomposite. It holds all advantages of chitosan while the high swelling size expansion and poor mechanical properties of chitosan are voided with using CNC and citric acid, crosslinker. Briefly, CNC nanoparticles induced bioactivity, high porosity, firm, oriented and uniform pore structures as well as appropriated mechanical properties and bone cell proliferation in the SC5 scaffold. HA was also used to mimic native bone composition in this study.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1. Materials and Methods

Materials and instruments. Plasma Biotal Ltd. UK provided CAPTAL® sintered type ‘S’ (5.6 μm) sintered and CAPTAL® unsintered type ‘R’ hydroxylapatite (3.66 μm) hydroxylapatites. Cellulose nanocrystal (CNC) used in the study was provided by InnoTech Alberta (IA), Edmonton, Alberta, Canada. Chitosan high molecular weight and other chemicals were purchased from Sigma-Aldrich, USA.

SEM microscope was Zeiss Sigma 300 VP-FESEM with environmental ionization. Samples were mounted on carbon film and SEM was conducted. ImageJ software was used to analyze all SEM images. Elemental analysis by energy-dispersive X-ray spectroscopy (EDS) analysis Bruker energy dispersive X-ray spectroscopy with dual silicon drift detectors with a resolution of 123 eV was used for this analysis.

Differential Scanning calorimetry (DSC) thermograms were recorded by the TA Q2000 DSC instrument. The heating rate was 5° C./min and the final temperature was 200° C. under nitrogen. The Thermo-Gravimetry Analysis (TGA) instrument was a Mettler Toledo TGA/DSC 1 STARe system using STARe software. The heating rate was 10° C./min under nitrogen gas. The temperature range was the ambient temperature to 200° C.

Assessing mechanical properties of scaffolds by the compression test instrument was conducted by a Tensile tester (Instron 5943, Instron, Norwood, Mass., USA) equipped with a 1 kN load cell. The diameter and height of all molded scaffolds were measured by a caliper before testing.

FT-IR instrument was a Nicolet 8700. KBR pellets were used. (Range—4000 cm⁻¹−450 cm⁻¹ Resolution—4.000 and number of Scans—32).

X-ray diffraction (XRD) was performed by Rigaku Ultimate IV instrument (Cu, 40 Kv, 44 mA, 20=5-60 degree, 2°/min) for the morphology and interaction between components measurements.

Differential Scanning calorimetry (DSC) thermograms were recorded by TA Q2000 DSC instrument. The heating rate was 5° C./min and the final temperature was 200° C. under nitrogen.

General steps for fabrication of bio-nanocomposites. 1) Preparation of chitosan solution; 2) adjustment of pH; 3) addition of prepared CNC solution; 4) addition of hydroxylapatite solution; 5) molding of biomaterial composition; 6) freeze-drying of mold; 7) addition of crosslinker; 8) heat treatment of molded composition; and 9) washing away excess reagents.

CNC preparation. Briefly, fully bleached wood pulp was hydrolyzed using 64% sulfuric acid at 45° C. for 2 hours and then neutralized by 5-8% sodium hydroxide. The salt was removed by diafiltration process, and the CNC suspension was spray dried. This process produces highly crystalline cellulose. CNC used in this study had a white powder appearance with the following characteristics for dry CNC: density of 1.6 g/cm³, crystallinity index of >80%. The dimensions, provided by (IA), are 5-15 nm in width, 100-200 nm in length, and 200-300 m²/g specific surface area. The estimated zeta potential was reported to be <−30 mV at pH 6.

Fabrication of bio-nanocomposites. Two series of scaffolds (in total 8) were fabricated with a freeze-drying method in the study. The first one called S series were fabricated as follows. Chitosan solution was prepared with 1% w/v concentration in an acidic medium. In a typical experiment, 0.5 g of chitosan was dissolved in 50 mL of dd water. 0.5 mL of HCl 5M was added to the mixture. The solution was stirred for 24 h. The pH of the solution was adjusted at 5-6 with 5% of the sodium bicarbonate solution. The solution was sonicated for a few minutes.

Different amounts of CNC (Table 2) in 10 mL of deionized distilled (dd) water sonicated for 15 minutes. CNC mixture was added to the solution of chitosan by a peristaltic pump dropwise with an adjusted slow speed while the solution of chitosan stirred vigorously. The resulted mixture was vigorously stirred for 24 hours. Different amounts of HA in 5 mL of dd water (Table 2) was added to the solution, dropwise, and stirred overnight. The solution was molded in a suitable mold and frozen in dry ice and acetone. The solid mixture was lyophilized for 48 hours. The second series of the scaffolds with citric acid called SC series were fabricated similarly to the procedure for the S series with use of 4% w/w of citric acid. The composition of each formulation is shown in Table 2.

Bioactivity evaluation. Molded porous scaffolds, 5 mm diameter and 15 mm height, as measured by means of a caliber, were soaked in 15 mL of simulated body fluid (SBF) prepared according to the reported protocol at 37° C. and pH 7.4. The solutions were refreshed once every two days. The scaffolds were kept in SBF solutions for 2, 7, and 14 days. After incubation, the scaffolds were soaked and rinsed by dd water for 10 minutes, then dried by a freeze-dryer. The amount of adsorption of the calcium phosphate on the scaffolds was measured and evaluated with gravimetric analysis and EDS-SEM.

Degradation study. Degradation of the bio-nanocomposite over time was measured by gravimetric analysis and morphological investigations using SEM. A piece of bio-nanocomposite with a certain size and weight was soaked in PBS (pH 7.4) containing 0.01% (w/v) sodium azide (added to prevent the growth of bacteria) at 37° C. for 7, 15, 30, 60 days with gentle shaking in a water bath. The percentage of the weight changes for the samples was measured and the morphology of the specimen was assessed using SEM.

Swelling in water and PBS buffer. The height and diameter of five different scaffolds were measured before and after swelling. Pre-weighed scaffolds (n=5) were immersed in 20 mL of dd water and left at room temperature in water or PBS. At different time points, the scaffold was then taken out of the water, wiped off with Kimwipe, and weighed. To measure the equilibrium swelling ratio, the scaffolds were immersed in dd water for 24 h; wiped off and weighed. The following equation was used to calculate equilibrium swelling. Equilibrium swelling of the scaffolds was measured in PBS at 37° C., as well. Equilibrium swelling was measured using the following equation:

$$Equilibrium swelling ( ES ) = \frac{M w - M d}{M d} * 100 ,$$

where M_{w} is wet weight and M_{d} is the dry weight of the scaffold.

Cytotoxicity using MTT assay in 2D culture. Two cell lines were utilized for cytotoxicity assay using MTT reagent as the indicator of cell metabolic activity. MG63 is a human bone osteosarcoma cell line that was grown in Eagle's Minimum Essential Medium (EMEM). MC3T3 is an osteoblast mouse cell line that was grown in (DMEM F:12). Both cell lines are adherent cells. Scaffolds were cut, weighed (0.5, 1, and 1.5 mg), and sterilized by UV irradiation for 4 hours.

The scaffold sample was placed on a 48 well plate. A 300 uL media was added to each well. Specified and equal numbers of cells were added to the bottom of each well. Three wells were selected with no scaffold as a control for 2D cell culture. After culturing cells with suspended scaffold at specific times, 75 uL of 5 mg/mL of MTT was added to each well. The culture plate was left for 2 hours in an incubator. The media was removed. Each well was washed with HEPES to remove the remaining media. A 300 uL of DMSO was added to each well and left for a few hours in dark. The supernatant of each well was transferred to another plate. The scaffold was washed with another 300 uL of DMSO. The plate was left for a few hours. Two DMSO solutions were added together. A plate reader was used to measure the concentration of purple MTT at 570 nm.

Cell viability using MTT assay in 3D culture. Two cell lines were used for cell proliferation assay using MTT reagent as the indicator of cell metabolic activity. MG63 is a human bone osteosarcoma cell line that was grown in Eagle's Minimum Essential Medium (EMEM). MC3T3 is an osteoblast mouse cell line that was grown in (DMEM F:12). Both cell lines are adherent cells. Scaffolds were cut in 3D shape and sterilized by UV irradiation for 4 hours.

The scaffold samples (3D cylindrical shape) were placed on a 48 well plate. Equal numbers of cells were added on the top of each scaffold. A 300 uL media was then added to each well. A scaffold with zero amount of CNC was selected as control. After culturing cells on 3D scaffolds for 5 days, 75 uL of 5 mg/mL of MTT was added to each well. The culture plate was left for 2 hours in an incubator. The media was removed. Each well was washed with HEPES to remove the remaining media. A 300 uL of DMSO was added to each well and left overnight with gently shaking in dark. The supernatant of each well was transferred to another plate. The scaffold was washed with another 300 uL of DMSO to remove the rest of the MTT from the scaffolds. The plate was left for a few hours to remove the entire MTT properly. Two of the DMSO solutions were added together. A plate reader was used to measure the concentration of purple MTT at 570 nm.

Assessing cell morphology by SEM. Cells grown in 3D culture were fixed using 4% formalin solution in 1×PBS for 1 hour in 4° C. The cells were then washed with PBS and dd water consecutively. The scaffolds containing cells were frozen in 5% DMSO and then freeze-dried. Various slices of the cross-section of the dried scaffolds were prepared by cutting the scaffolds. SEM was used to take images from the cross-section slices to assess and evaluate osteoconductivity, cell proliferation of scaffolds.

Statistical analysis. Data are presented as mean±standard deviation (SD) of measurements throughout the manuscript. Statistical significance of difference was tested one-way ANOVA test. The significance level (a) was set at 0.05.

TABLE 2
Formulation mass (g) compositions of bio-
nanocomposites prepared by casting method.

| | Original | Formulation + | CS | CNC | HA | |
|---|---|---|---|---|---|---|
| no. | Formulation | citric acid | (g) | (g) | (g) | CS:CNC |
| 1 | S5 | SC5 | 0.50 | 0.50 | 1.20 | 1:1 |
| 2 | S3 | SC3 | 0.50 | 0.30 | 0.95 | 0.5:1 |
| 3 | S1 | SC1 | 0.50 | 0.10 | 0.70 | 5:1 |
| 4 | S0 | SC0 | 0.50 | 0.00 | 0.60 | 0.5:0 |

### Example 2. Characterization of Scaffolds

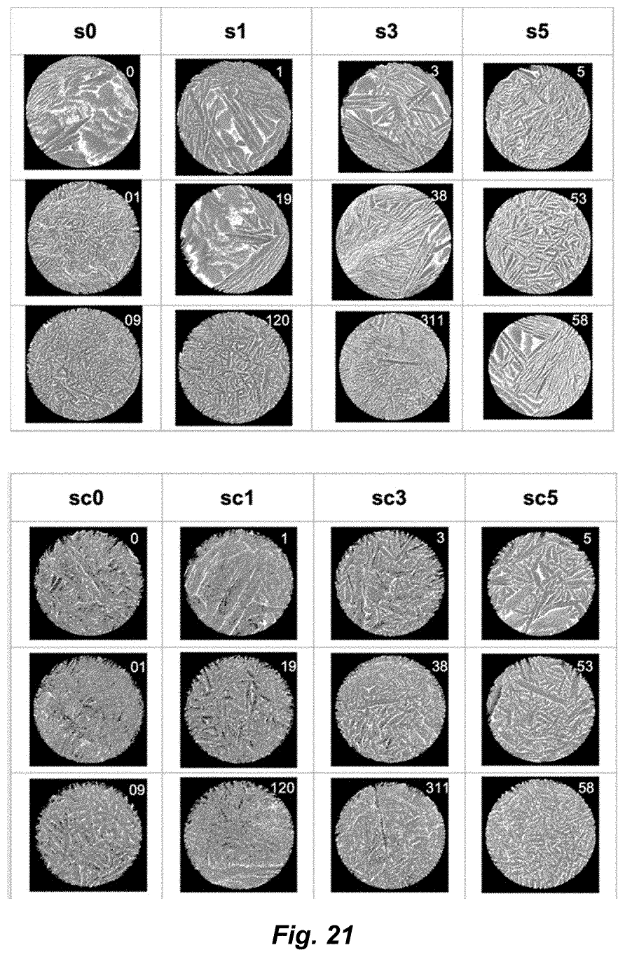

Micro-CT scanning was conducted on the modified (SC) and unmodified (S) scaffolds. The images of cross-sections of modified and unmodified scaffolds exhibited a porous structure in the fabricated scaffolds, as shown in FIG. 21. In addition, micro-CT images revealed a very low percent of closed pores for all series of modified and unmodified scaffolds (FIG. 22). The data showed that in S3 and S5 series, the closed percent porosity significantly reduced during modifications about 2-3 folds.

The connectivity density of the porous biomaterials with more CNC content (unmodified S3, S5, and modified SC3 and SC5) had higher values compared to lower CNC content scaffolds (S0, S1, SC0 and SC1). Therefore, higher CNC value in the biomaterials prompted connectivity of the biomaterials. The modification process did not change connectivity density significantly, as shown in FIG. 23.

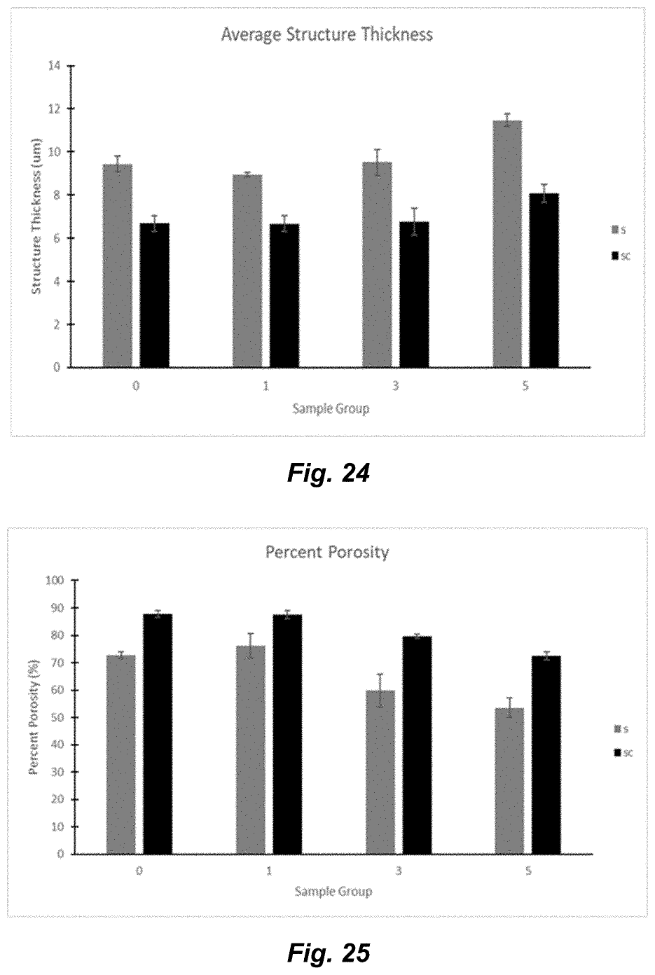

Structural thickness deceased about 2 μm with modification of the scaffolds. The crosslinking was carried out in the modification process. Thus, crosslinking can compact the fibres together. The Structural thickness shows in FIG. 24. In addition, the micro-CT results showed that the modification process improved total porosity, as shown in FIG. 25.

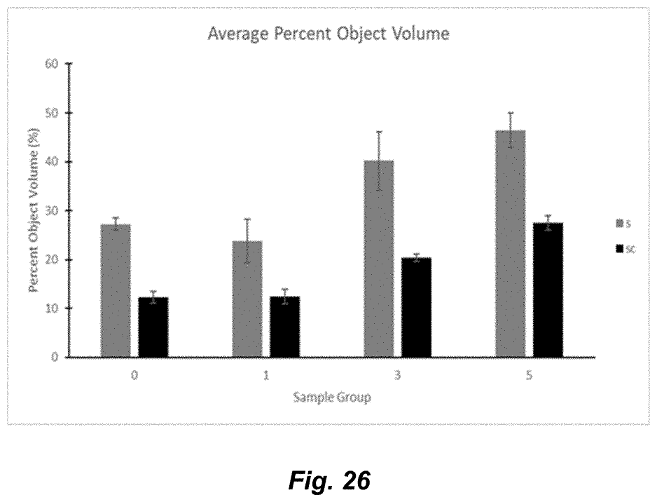

The average percent object volume deceased with modification, as shown in FIG. 26. The results agree well with the total porosity results. Remarkably, crosslinking formed compacted fibres, therefore the volume of fibres decreased with modification process.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A bio-nanocomposite comprising chitosan (CS), cellulose nanocrystals (CNC) and apatite (XA) wherein the mass ratio CS:CNC is about 0.5:1 to about 10:1, CS is crosslinked to CNC via a linker to form a polymer matrix (PM), and XA is uniformly dispersed in the polymer matrix wherein the mass ratio of XA:PM is about 1:1 to about 1.5:1.

2. The bio-nanocomposite of claim 1 wherein the mass ratio CS:CNC is about 1:1 to about 5:1.

3. The bio-nanocomposite of claim 1 wherein the mass ratio XA:PM is about 1.1:1 to about 1.2:1.

4. The bio-nanocomposite of claim 1 wherein XA comprises sintered hydroxylapatite (HA).

5. The bio-nanocomposite of claim 1 wherein the linker comprises an organic acid.

6. The bio-nanocomposite of claim 5 wherein the bio-nanocomposite comprises about 0.2% w/w to about 10% w/w of the organic acid.

7. The bio-nanocomposite of claim 5 wherein the organic acid is citric acid wherein the carboxyl groups on citric acid are covalently linked to the hydroxyl moieties on CNC via an ester bond and are covalently linked to the hydroxyl moieties on chitosan via an ester bond and/or the carboxyl groups on citric acid are covalently linked to the amine moieties on chitosan via an amide bond.

8. The bio-nanocomposite of claim 1 wherein CNC is an oxidized CNC comprising added carboxyl groups wherein the carboxyl groups are covalently linked to the hydroxyl moieties on chitosan via an ester bond, the carboxyl groups are covalently linked to the amine moieties on chitosan via an amide bond, or a combination thereof.

9. The bio-nanocomposite of claim 1 wherein the bio-nanocomposite comprises interconnected pores.

10. The bio-nanocomposite of claim 9 wherein the bio-nanocomposite has about 60% to about 90% porosity.

11. The bio-nanocomposite of claim 9 wherein the interconnected pores have an average pore diameter of about 10 micrometers to about 30 micrometers.

12. The bio-nanocomposite of claim 1 wherein the linker is citric acid, the apatite comprises sintered hydroxylapatite (HA), the mass ratio CS:CNC is about 1:1 to about 5:1, and the mass ratio HA:PM is about 1.1:1 to about 1.2:1.

13. The bio-nanocomposite of claim 12 wherein the bio-nanocomposite comprises interconnected pores and about 90% porosity, wherein the interconnected pores have an average pore diameter of about 25 micrometers to about 30 micrometers.

14. The bio-nanocomposite of claim 1 further comprising bone cells or cartilage cells.

15. The bio-nanocomposite of claim 1 wherein the bio-nanocomposite has a compression modulus at least two-times greater than a corresponding bio-nanocomposite that is not crosslinked; and the bio-nanocomposite has a weight loss of about 10% or less in phosphate buffered saline (PBS) S) at pH 7.4 at 37° C. after 30 days.

16. The bio-nanocomposite of claim 15 wherein the compression modulus is at least four-times greater than a corresponding bio-nanocomposite that is not crosslinked.

17. A method for forming a bio-nanocomposite artificial matrix for bone or cartilage comprising:
a) contacting chitosan (CS), cellulose nanocrystals (CNC), sintered hydroxylapatite (HA) and an aqueous solution at about pH 5 to about pH6 to form a mixture;
b) freezing the mixture in a cast and lyophilizing the frozen mixture to form a scaffold;
c) contacting the scaffold, citric acid, and an alcohol; and
d) removing excess citric acid and the alcohol;
wherein the mass ratio of CS:CNC is about 0.5:1 to about 10:1, CS is crosslinked to CNC via citric acid to form a polymer matrix (PM), and the mass ratio of HA:PM is about 1:1 to about 1.5:1;
wherein HA is uniformly dispersed in the polymer matrix and the artificial matrix is thereby formed.

18. The method of claim 17 wherein the artificial matrix comprises about 0.2% w/w to about 10% w/w of the citric acid.

19. The method of claim 17 wherein the artificial matrix comprises interconnected pores and about 90% porosity, wherein the interconnected pores have an average pore diameter of about 25 micrometers to about 30 micrometers.

20. The method of claim 17 further comprising contacting the artificial matrix, bone cells or cartilage cells, and optionally one or more growth factors, under suitable physiological conditions for bone or cartilage regeneration.
